(19) United States
(12) Patent Application Publication
(10) Pub. No.: US 2005/0038070 A1
(43) Pub. Date: Feb. 17, 2005

(54) Asthma and allergic inflammation modulators

(75) Inventors: Wayne D. Inman, Belmont, CA (US); Jiwen Liu, Foster City, CA (US); Julio C. Medina, San Carlos, CA (US); Shichang Miao, Foster City, CA (US); Hua Lucy Tang, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AMGEN INC.

(21) Appl. No.: 10/887,341

(22) Filed: Jul. 7, 2004

Related U.S. Application Data
(60) Provisional application No. 60/485,978, filed on Jul. 9, 2003.

(51) Int. Cl. A61K 31/47; C07D 215/38; A61K 31/4709

(52) U.S. Cl. 514/313; 546/159

(57) ABSTRACT

Compounds, pharmaceutical compositions and methods are provided that are useful in the treatment of inflammatory and immune-related diseases and conditions. In particular, the invention provides compounds which modulate the function and/or expression of proteins involved in atopic diseases, inflammatory conditions and cancer. The subject compounds are tetrahydroquinoline derivatives.

### CROSSED-REFERENCE TO RELATED APPLICATION

This application claims the benefit of Provisional Application Ser. No. 60/485,978, filed Jul. 9, 2003, the content of which is incorporated herein by reference.

### BACKGROUND OF THE INVENTION

G-protein coupled receptors play important roles in diverse signaling processes, including those involved in host defense mechanisms. Immune responses to infectious diseases, injury, tumors and organ transplantation and in diseases and conditions such as asthma, allergy, rheumatoid arthritis and neoplasia have been linked to GPCR regulation. Exaggerated or misdirected immune responses are responsible for many inflammatory and hypersensitivity diseases which, left untreated, can result in tissue or organ damage, pain and/or loss of function. Tissue inflammation is largely implicated in the pathogenesis of such diseases, of which asthma and allergic diseases are among the most well characterized. The mechanisms underlying airway inflammation and hyperreactivity are similar to those underlying allergic inflammation in other tissues, such as the skin and gut.

Prostaglandins are lipid-derived inflammatory mediators that recruit macrophages, T cells, eosinophils, basophils and neutrophils from peripheral blood to damaged or inflamed tissues. In addition, prostaglandins can, depending on the target cell type, induce or inhibit intracellular Ca²⁺ mobilization, cAMP production, platelet aggregation, leukocyte aggregation, T cell proliferation, lymphocyte migration, and Th2 cell chemotaxis, IL-1a and IL-2 secretion and vascular and non-vascular smooth muscle contraction in responsive cells. Prostaglandins have been implicated in fever, various allergic diseases, vascular and non-vascular smooth muscle relaxation, pain perception, sleep, platelet aggregation and reproductive processes. Prostaglandins exert their effects by interacting with specific GPCRs.

Prostaglandin D₂ (PGD₂) is the major inflammatory mediator released by activated mast cells, typically found near skin surfaces, mucous membranes and blood vessels, upon immunological challenge (Lewis et al. (1982) J. Immunol. 129:1627-1631). During asthma and allergic responses, PGD₂ is released in large amounts. The role of PGD₂ in the initiation and maintenance of allergic inflammation has been well established in mouse models of asthma. For example, it has been demonstrated that overproduction of PGD₂ in vivo by PGD₂ synthase exacerbates airway inflammation in a mouse model of asthma (Fujitani et al. (2002) J. Immunol. 168:443-449).

A PGD₂-selective receptor, designated DP, has been identified (Power et al. (1995) J. Biol. Chem. 270:19495-19500). In humans, DP is expressed in smooth muscle, platelets, small intestine and brain, and its expression in lung epithelium is induced by allergic challenge. Receptor activation induces cAMP production and intracellular Ca²⁺ mobilization, and is believed to inhibit platelet aggregation and cell migration and induce relaxation of various smooth muscles. DP is coupled primarily to Gαs protein.

Significantly, in an OVA induced asthma model, DP^{-/-} mice exhibited reduced asthma symptoms, e.g., reduced cellular infiltration of eosinophils and lymphocytes in BAL fluid, reduced Th2 cytokine levels in BAL fluid and reduced airway hyperreactivity to acetylcholine (Matsuoka et al. (2002) Science 287:2013-2019). The increased cellular infiltration in lung tissue and mucus secretion by airway epithelial cells characteristic of asthma in humans and observed in wild-type mice was not observed in DP-deficient mice.

Recently, an additional PGD₂-selective receptor, designated chemoattractant receptor-homologous molecule expressed on Th2 cells, or CRTH2, has been identified (Hirai et al. (2001) J. Exp. Med. 193(2):255-261). The receptor was previously referred to as GPR44 or DL1R. Among peripheral blood T lymphocytes, human CRTH2 is selectively expressed on Th2 cells, and is highly expressed on cell types associated with allergic inflammation such as eosinophils, basophils and Th2 cells. It has been shown that CRTH2 activation induces intracellular Ca²⁺ mobilization and infiltration of Th2 cells, eosinophils and basophils.

Protein sequence analysis indicates that CRTH2 has no significant homology to DP, but rather, is related to members of the N-formyl peptide receptor (FPR) subfamily (Nagata et al. (1999) J. Immunol. 162:1278-1286). In contrast to DP, CRTH2 has been shown to couple primarily to Gαi protein.

These observations suggest that CRTH2 and DP may function independently to regulate aspects of allergic inflammation.

The increasing incidence of asthma, allergic diseases and immunologic diseases worldwide underscores the need for new therapies to effectively treat or prevent these diseases. The discovery of small molecules that modulate CRTH2 and/or one or more other PGD₂ receptors is useful for the study of physiological processes mediated by CRTH2 and/or one or more other PGD₂ receptors and the development of therapeutic agents for asthma, allergic diseases and other immunologic diseases. Novel compounds which display such desirable activity are described herein.

### SUMMARY OF THE INVENTION

The invention provides compounds, pharmaceutical compositions and methods useful for treating or preventing conditions and disorders associated with allergic inflammation processes. In particular, the invention provides compounds, pharmaceutical compositions and methods useful for treating or preventing asthma, allergic diseases, inflammatory conditions and cancer.

Certain compounds of the invention have the general formula (I): 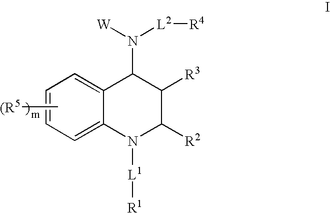
wherein W is aryl, heteroaryl, (C₁-C₈)alkyl or cyclo(C₃-C₈)alkyl; L¹ is C(O), SO₂ or (C₁-C₄)alkylene; L² is a single bond, C(O) or SO₂; R¹ is (C₁-C₈)alkyl, aryl, aryl(C₁-C₄)alkyl, aryl(C₁-C₄)alkoxy, aryl(C₁-C₄)alkenyl or heteroaryl; R² and R³ are independently hydrogen or (C₁-C₈)alkyl; and R⁴ is (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl.

Each R⁵ is independently halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ or —CH(Ph)₂; optionally, two adjacent R⁵ groups may be combined to form a 5-, 6-, 7- or 8-membered fused ring containing the carbon atoms to which they are attached and 0, 1 or 2 additional heteroatoms selected from N, O and S. R′, R− and R′″ are independently hydrogen, (C₁-C₈)alkyl, aryl, aryl(C₁-C₄)alkyl or heteroaryl; optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S. The subscript m is 0, 1, 2, 3 or 4.

Other compounds of the invention have the general formula (VI): 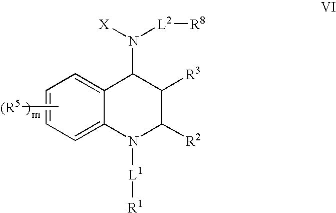
wherein X is hydrogen, (C₁-C₈)alkyl or aryl(C₁-C₄)alkyl; L¹ is C(O), SO₂ or (C₁-C₄)alkylene; L² is a single bond, C(O) or SO₂; R′ is (C₁-C₈)alkyl, aryl, aryl(C₁-C₄)alkyl, aryl(C₁-C₄)alkoxy or aryl(C₁-C₄)alkenyl; R² and R³ are independently hydrogen or (C₁-C₈)alkyl; and R⁸ is (C₁-C₈)alkyl, aryl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl group.

Each R⁵ is independently halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′ —NR″CO₂R′, —N(R′)C(O)NR″R′″, —NR′C(NH₂)═NR″, —S(O)R′, —SO₂R′, —SO₂NR′R″, —N₃ or —CH(Ph)₂; optionally, two adjacent R⁵ groups may be combined to form a 5-, 6-, 7- or 8-membered fused ring containing the carbon atoms to which they are attached and 0, 1 or 2 additional heteroatoms selected from N, O and S. R′, R″ and R″′ are independently hydrogen, (C₁-C₈)alkyl, aryl, aryl(C₁-C₄)alkyl or heteroaryl. optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S. The subscript m is 0, 1, 2, 3 or 4.

Within the above compounds of formula V¹, R⁸ is other than phenyl when X is hydrogen and L² is a single bond.

Still other compounds of the invention have the general formula (VII): 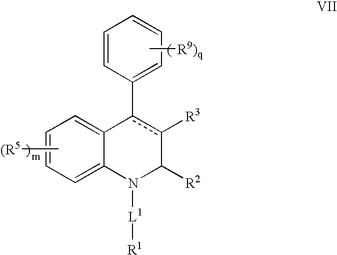
wherein L¹ is C(O), SO₂ or (C₁-C₄)alkylene; R¹ is (C₁-C₈)alkyl, aryl, aryl(C₁-C₄)alkyl, aryl(C₁-C₄)alkoxy, aryl(C₁-C₄)alkenyl or heteroaryl; R² and R³ are independently hydrogen or (C₁-C₈)alkyl and the dotted line indicates an optional bond.

Each R⁵ is independently halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ or —CH(Ph)₂; optionally, two adjacent R⁵ groups may be combined to form a 5-, 6-, 7- or 8-membered fused ring containing the carbon atoms to which they are attached and 0, 1 or 2 additional heteroatoms selected from N, O and S. Each R⁹ is independently halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, carboxy(C₁-C₄)alkyl, —CONR′R″, —C(O)R′, —OC(O)R′, —OC(O)NR′R″, —NR″C(O)R′, —NR″CO₂R′, —S(O)R′, —SO₂R′ or —SO₂NR′R″; optionally, two adjacent R⁹ groups may be combined to form a 5-, 6-, 7- or 8-membered fused ring containing the carbon atoms to which they are attached and 0, 1 or 2 additional heteroatoms selected from N, O and S. R′, R″ and R′″ are independently hydrogen, (C₁-C₈)alkyl, aryl, aryl(C₁-C₄)alkyl or heteroaryl; optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S. The subscript m is 0, 1, 2, 3 or 4 and the subscript q is 0, 1, 2, 3, 4 or 5.

Unless otherwise indicated, the compounds provided in the above formulae are meant to include pharmaceutically acceptable salts and prodrugs thereof.

The invention also provides pharmaceutical compositions comprising a compound of the invention and a pharmaceutically acceptable carrier, excipient or diluent.

The invention also provides methods for treating or preventing inflammatory conditions, immune disorders, asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease and cancer, comprising administering to a subject in need thereof a therapeutically effective amount of a compound of the invention.

The invention further provides methods for treating or preventing a condition or disorder responsive to modulation of CRTH2 and/or one or more other PGD₂ receptors, comprising administering to a subject in need thereof a therapeutically effective amount of a compound of the invention.

The invention also provides methods for treating or preventing a condition or disorder mediated by CRTH2 and/or one or more other PGD₂ receptors, comprising administering to a subject in need thereof a therapeutically effective amount of a compound of the invention.

The invention also provides methods for modulating CRTH2 and/or one or more other PGD₂ receptors, comprising contacting a cell with a compound of the invention.

Other objects, features and advantages of the invention will become apparent to those skilled in the art from the following description and claims.

### BRIEF DESCRIPTION OF THE DRAWINGS

FIGS. 1 and 2 provide the structures of exemplary compounds of the invention.

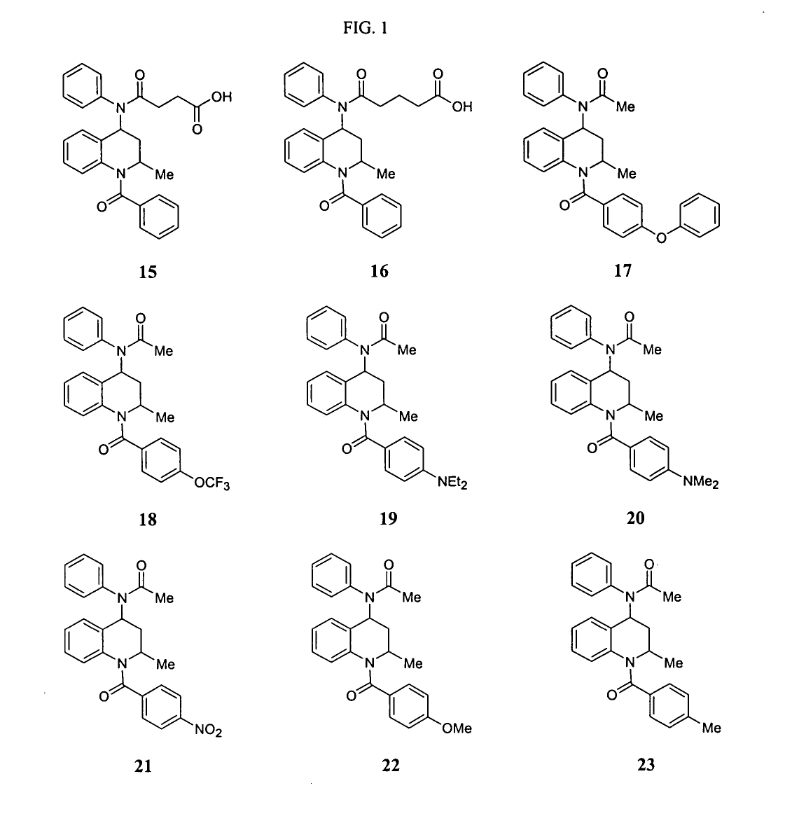

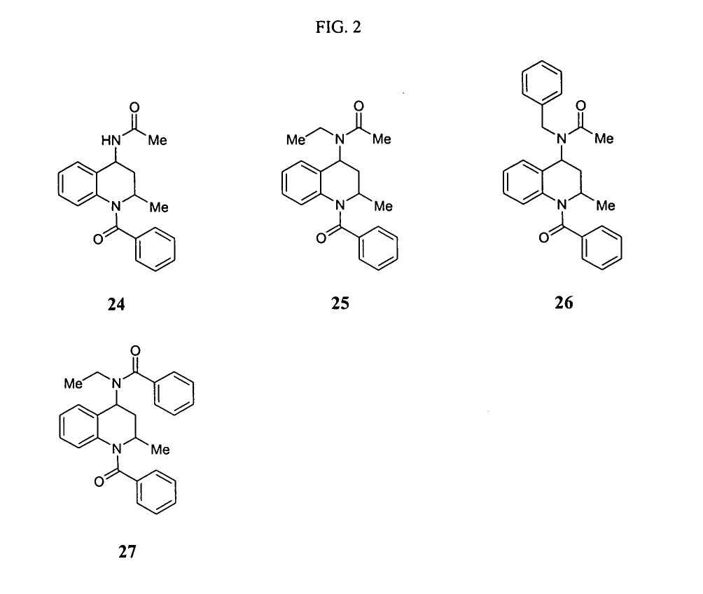

### DETAILED DESCRIPTION OF THE INVENTION

Abbreviations and Definitions

The abbreviations used herein are conventional, unless otherwise defined.

The terms “treat”, “treating” and “treatment”, as used herein, are meant to include alleviating or abrogating a disease and/or its attendant symptoms and alleviating or eradicating the cause of the disease itself.

The terms “prevent”, “preventing” and “prevention”, as used herein, refer to a method of delaying or precluding the onset of a disease and/or its attendant symptoms, barring a subject from acquiring a disease or reducing a subject's risk of acquiring a disease.

The term “therapeutically effective amount” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the term “CRTH2” refers to a CRTH2 protein (RefSeq Accession No. NP_{—}007469) or variant thereof that is capable of mediating a cellular response to PGD₂ in vitro or in vivo. CRTH2 variants include proteins substantially homologous to native CRTH2, i.e., proteins having one or more naturally or non-naturally occurring amino acid deletions, insertions or substitutions (e.g., CRTH2 derivatives, homologs and fragments). The amino acid sequence of CRTH2 variant preferably is at least about 80% identical to a native CRTH2, more preferably at least about 90% identical, and most preferably at least about 95% identical.

As used herein, the terms “other PGD₂ receptor”, “another PGD₂ receptor” and the like refer to a prostanoid receptor protein other than CRTH2, or variant thereof, that is capable of mediating a cellular response to PGD₂ in vitro or in vivo. Another PGD₂ receptor may be selective for PGD₂, e.g., DP (RefSeq Accession No. NP_{—}000944), or other one or more other prostanoids (e.g., EP₁, EP₂, EP₃ and EP₄, FP, IP and TP). Other PGD₂ receptor variants include proteins substantially homologous to a corresponding native prostanoid receptor other than CRTH2, i.e., proteins having one or more naturally or non-naturally occurring amino acid deletions, insertions or substitutions (e.g., derivatives, homologs and fragments of another PGD₂ receptor). The amino acid sequence of other PGD₂ receptor variants preferably is at least about 80% identical to the corresponding native other PGD₂ receptors, more preferably at least about 90% identical, and most preferably at least about 95% identical. Preferably, another PGD₂ receptor is DP.

The terms “modulate”, “modulation” and the like refer to the ability of a compound to increase or decrease the function and/or expression of CRTH2 and/or one or more other PGD₂ receptors, where such function may include transcription regulatory activity and/or protein-binding. Modulation may occur in vitro or in vivo. Modulation, as described herein, includes the inhibition, antagonism, partial antagonism, activation, agonism or partial agonism of a function or characteristic associated with CRTH2 and/or one or more other PGD₂ receptors, either directly or indirectly, and/or the upregulation or downregulation of the expression of CRTH2 and/or one or more other PGD₂ receptors, either directly or indirectly. In a preferred embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction. The ability of a compound to inhibit the function of CRTH2 and/or one or more other PGD₂ receptors can be demonstrated in a biochemical assay, e.g., binding assay, or a cell-based assay, e.g., a transient transfection assay.

The term “CRTH2-modulating amount” refers to that amount of a compound that is needed to produce a desired effect in any one of the cell-based assays, biochemical assays or animal models described herein or otherwise known to the skilled artisan. Typically, a CRTH2-modulating amount of a compound will be at least that amount which exhibits an EC₅₀ in a reporter-gene cell-based assay (relative to an untreated control).

As used herein, the terms “CRTH2-responsive condition or disorder”, “condition or disorder responsive to CRTH2” and related terms and phrases refer to a condition or disorder associated with inappropriate, e.g., less than or greater than normal, CRTH2 activity and at least partially responsive to or affected by CRTH2 modulation (e.g., a CRTH2 antagonist or agonist results in some improvement in patient well-being in at least some patients). Inappropriate CRTH2 functional activity might arise as the result of CRTH2 expression in cells which normally do not express CRTH2, increased CRTH2 expression or degree of intracellular activation (leading to, e.g., inflammatory and immune-related disorders and diseases) or decreased CRTH2 expression. A CRTH2-associated condition or disorder may include a CRTH2-mediated condition or disorder.

As used herein, the phrases “CRTH2-mediated condition or disorder”, “a condition or disorder mediated by CRTH2” and related phrases and terms refer to a condition or disorder characterized by inappropriate, e.g., less than or greater than normal, CRTH2 activity. Inappropriate CRTH2 functional activity might arise as the result of CRTH2 expression in cells which normally do not express CRTH2, increased CRTH2 expression or degree of intracellular activation (leading to, e.g., inflammatory and immune-related disorders and diseases) or decreased CRTH2 expression. A CRTH2-mediated condition or disorder may be completely or partially mediated by inappropriate CRTH2 functional activity. However, a CRTH2-mediated condition or disorder is one in which modulation of CRTH2 results in some effect on the underlying condition or disorder (e.g., an CRTH2 antagonist or agonist results in some improvement in patient well-being in at least some patients).

The term “PGD₂ receptor-modulating amount” and related terms and phrases refer to that amount of a compound that is needed to produce a desired effect in any one of the cell-based assays, biochemical assays or animal models described herein or otherwise known to the skilled artisan. Typically, a PGD₂ receptor-modulating amount of a compound will be at least that amount which exhibits an EC₅₀ in a reporter-gene cell-based assay (relative to an untreated control).

As used herein, the term “condition or disorder responsive to another PGD₂ receptor” and related terms and phrases refer to a condition or disorder associated with inappropriate, e.g., less than or greater than normal, activity of another PGD₂ receptor and at least partially responsive to or affected by modulation of another PGD₂ receptor (e.g., another PGD₂ receptor antagonist or agonist results in some improvement in patient well-being in at least some patients). Inappropriate functional activity of another PGD₂ receptor might arise as the result of expression of another PGD₂ receptor in cells which normally do not express the receptor, increased expression of another PGD₂ receptor or degree of intracellular activation (leading to, e.g., inflammatory and immune-related disorders and diseases) or decreased expression of another PGD₂ receptor. A condition or disorder associated with another PGD₂ receptor may include a condition or disorder mediated by another PGD₂ receptor.

As used herein, the phrase “condition or disorder mediated by another PGD₂ receptor” and related phrases and terms refer to a condition or disorder characterized by inappropriate, e.g., less than or greater than normal, activity of another PGD₂ receptor. Inappropriate functional activity of another PGD₂ receptor might arise as the result of expression of another PGD₂ receptor in cells which normally do not express the receptor, increased expression of another PGD₂ receptor or degree of intracellular activation (leading to, e.g., inflammatory and immune-related disorders and diseases) or decreased expression of another PGD₂ receptor. A condition or disorder mediated by another PGD₂ receptor may be completely or partially mediated by inappropriate functional activity of another PGD₂ receptor. However, a condition or disorder mediated by of another PGD₂ receptor is one in which modulation of another PGD₂ receptor results in some effect on the underlying condition or disorder (e.g., another PGD₂ receptor antagonist or agonist results in some improvement in patient well-being in at least some patients).

The term “alkyl,” by itself or as part of another substituent, means, unless otherwise stated, a straight or branched chain, or cyclic hydrocarbon radical, or combination thereof, which is fully saturated, having the number of carbon atoms designated (i.e., C₁-C₈ means one to eight carbons). Examples of alkyl groups include methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl and the like.

The term “alkenyl”, by itself or as part of another substituent, means a straight or branched chain, or cyclic hydrocarbon radical, or combination thereof, which may be mono- or polyunsaturated, having the number of carbon atoms designated (i.e., C₂-C₈ means two to eight carbons) and one or more double bonds. Examples of alkenyl groups include vinyl, 2-propenyl, crotyl, 2-isopentenyl, 2-(butadienyl), 2,4-pentadienyl, 3-(1,4-pentadienyl) and higher homologs and isomers thereof.

The term “alkynyl”, by itself or as part of another substituent, means a straight or branched chain hydrocarbon radical, or combination thereof, which may be mono- or polyunsaturated, having the number of carbon atoms designated (i.e., C₂-C₈ means two to eight carbons) and one or more triple bonds. Examples of alkynyl groups include ethynyl, 1- and 3-propynyl, 3-butynyl and higher homologs and isomers thereof.

The term “alkylene” by itself or as part of another substituent means a divalent radical derived from alkyl, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Similarly, the term dialkylamino refers to an amino group having two attached alkyl groups that can be the same or different.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—SO₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O-Si(CH₃)₃. When a prefix such as (C₂-C₈) is used to refer to a heteroalkyl group, the number of carbons (2-8, in this example) is meant to include the heteroatoms as well. For example, a C₂-heteroalkyl group is meant to include, for example, —CH₂OH (one carbon atom and one heteroatom replacing a carbon atom) and —CH₂SH. The term “heteroalkylene” by itself or as part of another substituent means a divalent radical derived from heteroalkyl, as exemplified by —CH₂—CH₂—S—CH₂CH₂— and —CH₂—S—CH₂CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Thus, the terms “cycloalkyl” and “heterocycloalkyl” are meant to be included in the terms “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl”, are meant to include alkyl substituted with halogen atoms which can be the same or different, in a number ranging from one to (2m′+1), where m′ is the total number of carbon atoms in the alkyl group. For example, the term “halo(C₁-C₄)alkyl” is meant to include trifluoromethyl, 2,2,2-trifluoroethyl, 4-chlorobutyl, 3-bromopropyl, and the like. Thus, the term “haloalkyl” includes monohaloalkyl (alkyl substituted with one halogen atom) and polyhaloalkyl (alkyl substituted with halogen atoms in a number ranging from two to (2m′+1) halogen atoms). The term “perhaloalkyl” means, unless otherwise stated, alkyl substituted with (2m′+1) halogen atoms, where m′ is the total number of carbon atoms in the alkyl group. For example, the term “perhalo(C₁-C₄)alkyl”, is meant to include trifluoromethyl, pentachloroethyl, 1,1,1-trifluoro-2-bromo-2-chloroethyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from one to four heteroatoms selected from the group consisting of N, O and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 2-pyrimidinyl, 4-pyrimidinyl, 5-pyrimidinyl, 3-pyridazinyl, 4-pyridazinyl, 5-benzothiazolyl, purinyl, 2-benzimidazolyl, 5-indolyl, 1H-indazolyl, carbazolyl, α-carbolinyl, β-carbolinyl, γ-carbolinyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl and 8-quinolyl.

Preferably, the term “aryl” refers to a phenyl or naphthyl group which is unsubstituted or substituted. Preferably, the term “heteroaryl” refers to a pyrrolyl, pyrazolyl, imidazolyl, pyrazinyl, oxazolyl, isoxazolyl, thiazolyl, furyl, thienyl, pyridyl, pyrimidyl, pyrimidinyl, pyridazinyl, benzothiazolyl, purinyl, benzimidazolyl, indolyl, indazolyl, carbazolyl, carbolinyl, isoquinolyl, quinoxalinyl or quinolyl group which is unsubstituted or substituted.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical, unless otherwise indicated. Preferred substituents for each type of radical are provided below.

Substituents for the alkyl and heteroalkyl radicals (as well as those groups referred to as alkylene, alkenyl, heteroalkylene, heteroalkenyl, alkynyl, cycloalkyl, heterocycloalkyl, cycloalkenyl and heterocycloalkenyl) can be a variety of groups selected from: —OR′, =0, =NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′-C(O)NR″R′″, —NR′-SO₂NR″R′″, —NR″CO₂R′, —NH—C(₂)═NH, —NR′C(NH₂)═NH, —NH—C(NH₂)═NR′, —S(O)R′, —SO₂R′, —SO₂NR′R″, —NR″SO₂R, —CN and —NO₂, in a number ranging from zero to three, with those groups having zero, one or two substituents being particularly preferred. R′, R″ and R′″ each independently refer to hydrogen, unsubstituted (C₁-C₈)alkyl and heteroalkyl, unsubstituted aryl, aryl substituted with one to three halogens, unsubstituted alkyl, alkoxy or thioalkoxy groups, or aryl-(C₁-C₄)alkyl groups. When R′ and R″ or R″ and R′″ are attached to the same nitrogen atom, they can be combined with the nitrogen atom to form a 5-, 6- or 7-membered ring. For example, —NR′R″ is meant to include 1-pyrrolidinyl and 4-morpholinyl. Typically, an alkyl or heteroalkyl group will have from zero to three substituents, with those groups having two or fewer substituents being preferred in the present invention. More preferably, an alkyl or heteroalkyl radical will be unsubstituted or monosubstituted. Most preferably, an alkyl or heteroalkyl radical will be unsubstituted. From the above discussion of substituents, one of skill in the art will understand that the term “alkyl” is meant to include groups such as trihaloalkyl (e.g., —CF₃ and —CH₂CF₃).

Preferred substituents for the alkyl and heteroalkyl radicals are selected from: —OR′, —O, —NR′R″, —SR′, halogen, —SiR′R″R′″, —OC(O)R′, —C(O)R′, —CO₂R′, —CONR′R″, —OC(O)NR′R″, —NR″C(O)R′, —NR″CO₂R′, —NR′-SO₂NR″R′″, —S(O)R′, —SO₂R′, —SO₂NR′R″, —NR″SO₂R, —CN and —NO₂, where R′, R″ and R′″ are as defined above. Further preferred substituents are selected from: —OR′, —O, —NR′R″, halogen, —OC(O)R′, —CO₂R′, —CONR′R″, —OC(O)NR′R″, —NR″C(O)R′, —NR″CO₂R′, —NR′-SO₂NR″R′″, —SO₂R′, —SO₂NR′R″, —NR″SO₂R, —CN and —NO₂.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″CO₂R′, —NR′-C(O)NR″R′″, —NH—C(NH₂)═NH, —NR′C(NH₂)═NH, —NH—C(NH₂)═NR′, —S(O)R′, —SO₂R′, -SO₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ-B-, wherein A and B are independently —CH₂—, —O—, —NH—, —S—, —S(O)—, —SO₂—, —SO₂NR′- or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)₁-, where s and t are independently integers of from 0 to 3, and X is —O—, —NR′-, —S—, —S(O)—, —SO₂—, or —SO₂NR′-. The substituent R′ in —NR′- and —SO₂NR′— is selected from hydrogen or unsubstituted (C₁-C₆)alkyl. Otherwise, R′ is as defined above.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfric, hydriodic or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al. (1977) J. Pharm. Sci. 66:1-19). Certain specific compounds of the invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. Prodrugs of the compounds described herein are those compounds that readily undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. Additional examples include peptidyl derivatives of a compound of the invention.

Certain compounds of the invention can exist in unsolvated forms as well as solvated forms, including hydrated forms. In general, the solvated forms are equivalent to unsolvated forms and are intended to be encompassed within the scope of the invention. Certain compounds of the invention may exist in multiple crystalline or amorphous forms. In general, all physical forms are equivalent for the uses contemplated by the invention and are intended to be within the scope of the invention.

Certain compounds of the invention possess asymmetric carbon atoms (optical centers) or double bonds; the racemates, enantiomers, diastereomers, geometric isomers and individual isomers are all intended to be encompassed within the scope of the invention. These isomers can be resolved or asymmetrically synthesized using conventional methods to render the isomers “optically pure”, i.e., substantially free of its other isomers.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabled compounds are useful as therapeutic or prophylactic agents, e.g., cancer therapeutic agents, research reagents, e.g., CRTH2 assay reagents, and diagnostic agents, e.g., in vivo imaging agents. All isotopic variations of the compounds of the invention, whether radioactive or not, are intended to be encompassed within the scope of the invention.

Embodiments of the Invention

A class of compounds that modulate CRTH2 and/or one or more other PGD₂ receptors has been discovered. Depending on the biological environment (e.g., cell type, pathological condition of the host, etc.), these compounds can activate or inhibit the actions of CRTH2 and/or one or more other PGD₂ receptors (e.g., ligand binding). By activating or inhibiting CRTH2 and/or one or more other PGD₂ receptors, the compounds will find use as therapeutic agents capable of modulating diseases and conditions responsive to modulation of CRTH2 and/or one or more other PGD₂ receptors and/or mediated by CRTH2 and/or one or more other PGD₂ receptors. As noted above, examples of such diseases and conditions include inflammatory conditions, immune disorders, asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease and cancer. Additionally, the compounds are useful for the treatment and/or prevention of complications of these diseases and disorders (e.g., cardiovascular disease).

While the compounds of the invention are believed to exert their effects by interacting with CRTH2, the mechanism of action by which the compounds act is not a limiting embodiment of the invention. For example, compounds of the invention may interact with PGD₂ receptor subtypes other than CRTH2, e.g., DP receptor, and/or other prostanoid receptors, e.g., thromboxane A₂ (TXA₂) receptor. Indeed, as alluded to above, the present invention specifically contemplates the use of the disclosed compounds to modulate one or more PGD₂ receptors other than CRTH2.

Compounds contemplated by the invention include, but are not limited to, the exemplary compounds provided herein.

Compounds

In one aspect, the invention provides compounds of formula (I): 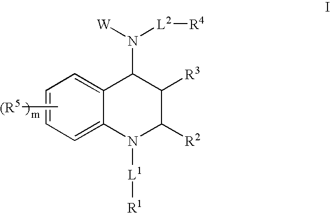
or a pharmaceutically acceptable salt or prodrug thereof. In formula I, the letter W represents an aryl, heteroaryl, (C₁-C₈)alkyl or cyclo(C₃-C₈)alkyl group. Exemplary W groups are phenyl and 4-chlorophenyl.

The symbol L¹ represents a divalent linkage selected from C(O), SO₂ and (C₁-C₄)alkylene. Exemplary L¹ groups are C(O), SO₂ and methylene.

The symbol L² represents a divalent linkage selected from a single bond, C(O) and SO₂. An exemplary L² group is C(O).

The substituent R¹ represents an (C₁-C₈)alkyl, aryl, aryl(C₁-C₄)alkyl, aryl(C₁-C₄)alkoxy, aryl(C₁-C₄)alkenyl or heteroaryl group. Exemplary R¹ groups are phenyl, 3-nitrophenyl, 4-fluorophenyl, n-butyl, styryl, benzyl, benzyloxy, 4-chlorophenyl, 4-methylphenyl, 4-methoxyphenyl, 3,4-dimethylphenyl, 3-methylphenyl, 4-tert-butylphenyl, 4-nitrophenyl, 4-aminophneyl, 3-fluoro-4-trifluoromethylphenyl, 4-dimethylaminophenyl, 4-diethylaminophenyl, 4-trifluoromethoxyphenyl and 4-phenyoxyphenyl.

The substituents R² and R³ independently represent hydrogen or a (C₁-C₈)alkyl group. An exemplary R² group is methyl. An exemplary R³ group is hydrogen.

The substituent R⁴ represents a (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl group. Exemplary R⁴ groups are methyl, ethyl, n-butyl, isobutyl, benzyl, 2-phenylethyl, 2-cyclopentylethyl, hydroxymethyl, methoxymethyl, ethoxymethyl, isopropyloxymethyl, ethylaminomethyl, —CH₂CO₂Me, —CH₂CH₂CO₂Me, —CH₂CH₂CH₂CO₂Me, carboxymethyl, 2-carboxyethyl, 3-carboxypropionyl, carbamoylmethyl, 2-carbamoylethyl, 2-carboxyethenyl and 3-carboxyphenyl.

Each substituent R⁵ independently represents a halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ or —CH(Ph)₂ group.

The substituents R′, R″ and R′″ independently represent hydrogen, (C₁-C₈)alkyl, aryl, aryl(C₁-C₄)alkyl or heteroaryl.

The subscript m is 0, 1, 2, 3 or 4.

In optional embodiments, two adjacent R⁵ groups may be combined to form a 5-, 6-, 7- or 8-membered fused ring containing the carbon atoms to which they are attached and 0, 1 or 2 additional heteroatoms selected from N, O and S and, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S.

Within formula I are provided several groups of embodiments, described below.

In one group of embodiments, W is aryl or heteroaryl. Within this group of embodiments, W is selected from the group consisting of phenyl, naphthyl, biphenyl, pyrrolyl, pyrazolyl, imidazolyl, pyrazinyl, oxazolyl, isoxazolyl, thiazolyl, furyl, thienyl, pyridyl, pyrimidyl, pyrimidinyl, pyridazinyl, benzothiazolyl, purinyl, benzimidazolyl, indolyl, indazolyl, carbazolyl, carbolinyl, isoquinolyl, quinoxalinyl and quinolyl. In one embodiment W is phenyl.

In another group of embodiments, L² is C(O). In one embodiment L² is C(O) and W is phenyl.

Another group of embodiments is represented by the formula (II): 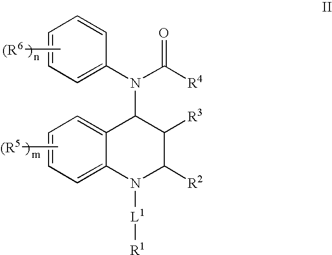
wherein each R⁶ is independently halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —S(O)R′, —SO₂R′ or —SO₂NR′R″ and the subscript n is 0, 1, 2, 3, 4 or 5. Optionally, two adjacent R⁶ groups may be combined to form a 5-, 6-, 7- or 8-membered fused ring containing the carbon atoms to which they are attached and 0, 1 or 2 additional heteroatoms selected from N, O and S. The variables L¹, R¹, R², R³, R⁴, R⁵, R′ and R″ and the subscript m have the meanings and groupings provided above.

In another group of embodiments of formula I, when R⁴ is unsubstituted alkyl, L² is other then C(O).

In another group of embodiments of formula I, R⁴ is substituted (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl. In one embodiment, R⁴ is aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl. In another embodiment, R⁴ is aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl and L² is C(O). In another embodiment, R⁴ is carboxy(C₁-C₄)alkyl, (C₁-C₄)alkoxycarbonyl(C₁-C₄)alkyl or carbamoyl(C₁-C₄)alkyl. In still another embodiment, R⁴ is carboxy(C₁-C₄)alkyl, (C₁-C₄)alkoxycarbonyl(C₁-C₄)alkyl or carbamoyl(C₁-C₄)alkyl and L² is C(O).

In another group of embodiments of formula I, R¹ is (C₁-C₈)alkyl, phenyl, naphthyl, aryl(C₁-C₄)alkyl, aryl(C₁-C₄)alkoxy, aryl(C₁-C₄)alkenyl or heteroaryl. In one embodiment, R¹ is phenyl or naphthyl. In another embodiment, R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy. In another embodiment, R¹ is phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

In another group of embodiments of formula I, L¹ is C(O). In one embodiment, L¹ is C(O) and R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy. In another embodiment, L¹ is C(O) and R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy and R⁴ is substituted (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl. In still another embodiment, L¹ is C(O) and R¹ is phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy, R⁴ is substituted (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl and L² is C(O).

Another group of embodiments is represented by the formula (III): 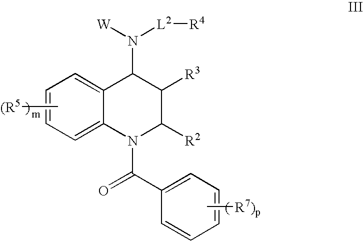
wherein each R⁷ is independently halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl or aryloxy and the subscript p is 0, 1, 2, 3, 4 or 5. Optionally, two adjacent R⁷ groups may be combined to form a 5-, 6-, 7- or 8-membered fused ring containing the carbon atoms to which they are attached and 0, 1 or 2 additional heteroatoms selected from N, O and S. The variables W, L², R², R³, R⁴, R⁵, R′ and R″ and the subscript m have the meanings and groupings provided above.

In one embodiment of formula III, the subscript p is 1. Another embodiment is represented by the formula (IV):

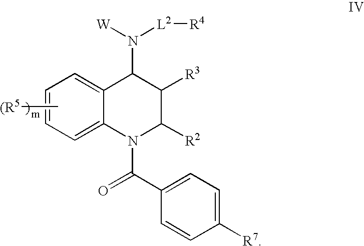

In another group of embodiments of formula I, R³ is hydrogen.

Another group of embodiments of formula I is represented by the formula (V): 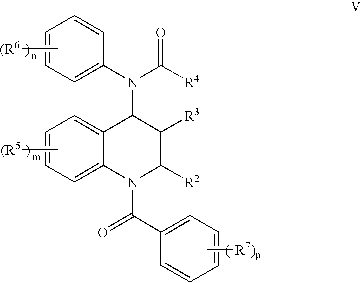
wherein the variables R², R³, R⁴, R⁵, R⁶, R⁷, R′ and R″ and the subscripts m, n and p have the meanings and groupings provided above.

The invention also provides compounds of formula (VI): 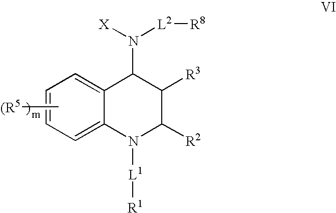
or a pharmaceutically acceptable salt or prodrug thereof. In formula VI, the letter X represents hydrogen or a (C₁-C₈)alkyl or aryl(C₁-C₄)alkyl group. Exemplary X groups are hydrogen, ethyl and benzyl.

The symbol L¹ represents a divalent linkage selected from C(O), SO₂ and (C₁-C₄)alkylene. An exemplary L¹ group is C(O).

The symbol L² represents a divalent linkage selected from a single bond, C(O) and SO₂. An exemplary L group is C(O).

The substituent R¹ represents an (C₁-C₈)alkyl, aryl, aryl(C₁-C₄)alkyl, aryl(C₁-C₄)alkoxy, aryl(C₁-C₄)alkenyl group or heteroaryl. An exemplary R¹ group is phenyl.

The substituents R² and R³ independently represent hydrogen or a (C₁—C₈)alkyl group. An exemplary R² group is methyl. An exemplary R³ group is hydrogen.

The substituent R⁸ represents a (C₁-C₈)alkyl, aryl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl or carboxy(C₂-C₄)alkenyl group. Exemplary R⁸ groups are methyl and phenyl.

Each substituent R⁵ independently represents a halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″,—C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ or —CH(Ph)₂ group.

The substituents R′, R″ and R′″ independently represent hydrogen, (C₁-C₈)alkyl, aryl, aryl(C₁-C₄)alkyl or heteroaryl.

The subscript m is 0, 1, 2, 3 or 4.

In optional embodiments, two adjacent R⁵ groups may be combined to form a 5-, 6-, 7- or 8-membered fused ring containing the carbon atoms to which they are attached and 0, 1 or 2 additional heteroatoms selected from N, O and S and, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S.

Within the above compounds of formula V¹, R⁸ is other than phenyl when X is hydrogen and L² is a single bond.

Within formula VI are provided several groups of embodiments, described below.

In one group of embodiments, L² is C(O).

In another group of embodiments, R⁸ is (C₁-C₈)alkyl or aryl. In one embodiment R⁸ is (C₁-C₈)alkyl or aryl and L² is C(O).

In another group of embodiments, R¹ is (C₁-C₈)alkyl, phenyl, naphthyl, aryl(C₁-C₄)alkyl, aryl(C₁-C₄)alkoxy, aryl(C₁-C₄)alkenyl or heteroaryl. In one embodiment, R¹ is phenyl or naphthyl. In another embodiment, R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

In another group of embodiments, L¹ is C(O). In one embodiment L¹ is C(O) and R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

In another group of embodiments, R³ is hydrogen.

The invention further provides compounds of formula (VII): 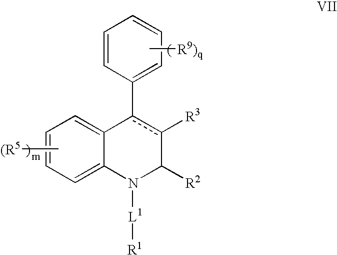
or a pharmaceutically acceptable salt or prodrug thereof. In formula VII, the symbol L¹ represents a divalent linkage selected from C(O), SO₂ and (C₁-C₄)alkylene. An exemplary L¹ group is C(O).

The substituent R¹ represents an (C₁-C₈)alkyl, aryl, aryl(C₁-C₄)alkyl, aryl(C₁-C₄)alkoxy, aryl(C₁-C₄)alkenyl group or heteroaryl. An exemplary R¹ group is phenyl.

The substituents R² and R³ independently represent hydrogen or a (C₁-C₈)alkyl group. An exemplary R² group is methyl. An exemplary R³ group is hydrogen.

Each substituent R⁵ independently represents a halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR″C(NH₂)═NR″, —S(O)R′, —SO₂R′, —SO₂NR′R″, —N₃ or —CH(Ph)₂.

Each substituent R⁹ independently represents a halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, carboxy(C₁-C₄)alkyl, —CONR′R″, —C(O)R′, —OC(O)R′, —OC(O)NR′R″, —NR″C(O)R′, —NR″CO₂R′, —S(O)R′, —SO₂R′ or —SO₂NR′R″ group. An exemplary R⁹ group is 3-methoxy.

The substituents R′, R″ and R′″ independently represent hydrogen, (C₁-C₈)alkyl, aryl, aryl(C₁-C₄)alkyl or heteroaryl.

The subscript m is 0, 1, 2, 3 or 4 and the subscript q is 0, 1, 2, 3, 4 or 5.

The dotted line indicates an optional bond. In other optional embodiments, two adjacent R⁵ or R⁹ groups may be combined to form a 5-, 6-, 7- or 8-membered fused ring containing the carbon atoms to which they are attached and 0, 1 or 2 additional heteroatoms selected from N, O and S and, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S.

Within formula VII are provided several groups of embodiments, described below.

In another group of embodiments, R¹ is (C₁-C₈)alkyl, phenyl, naphthyl, aryl(C₁-C₄)alkyl, aryl(C₁-C₄)alkoxy, aryl(C₁-C₄)alkenyl or heteroaryl. In one embodiment, R¹ is phenyl or naphthyl. In another embodiment, R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

In another group of embodiments, L¹ is C(O). In one embodiment L¹ is C(O) and R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

Another group of embodiments is represented by the formula (VIII): 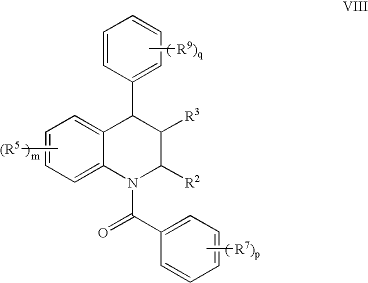
wherein each R⁷ is independently halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl or aryloxy and the subscript p is 0, 1, 2, 3, 4 or 5. Optionally, two adjacent R⁷ groups may be combined to form a 5-, 6-, 7- or 8-membered fused ring containing the carbon atoms to which they are attached and 0, 1 or 2 additional heteroatoms selected from N, O and S. The variables R², R³, R⁵, R⁹, R′ and R″ and the subscripts m and q have the meanings and groupings provided above.

In another group of embodiments of formula VII, R³ is hydrogen.

The compounds of the invention feature a 1,2,3,4-tetrahydroquinoline-derived ring, minimally substituted at the 1- and 4-positions. The numbering system used herein is illustrated below for the core ring system:

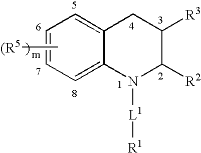

One of skill in the art will understand that formulas I, VI and VII each encompass two diastereomers, having relative structural orientations shown below (for formula I):

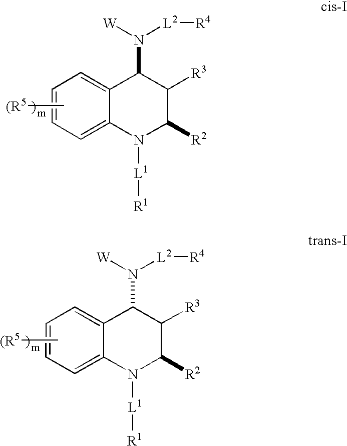

The invention encompasses novel compounds, novel pharmaceutical compositions and/or novel methods of use. While some compounds disclosed herein are available from commercial sources, the pharmaceutical compositions or methods of using these compounds are novel. Unless otherwise indicated, it is to be understood that the invention includes those compounds that are novel, as well as pharmaceutical compositions, various methods (e.g., methods of treating or preventing certain conditions and diseases mediated by CRTH2 and/or one or more other PGD₂ receptors), and the like which include both the novel compounds of the invention and compounds that are commercially available. Exemplary commercially available compounds of formula I include:
N-(1-benzoyl-1,2,3,4-tetrahydro-2-methyl-4-quinolinyl)-2-methyl-N-phenylpropanamide,
N-[1-(3-fluorobenzoyl)-1,2,3,4-tetrahydro-2-methyl-4-quinolinyl]-N-phenylhexanamide,
N-[1-(4-ethylbenzoyl)-1,2,3,4-tetrahydro-2,8-dimethyl-4-quinolinyl]-N-(2-methylphenyl)-2-(2-naphthalenyloxy)acetamide,
N-phenyl-N-[1,2,3,4-tetrahydro-2-methyl-1-(3-nitrobenzoyl)-4-quinolinyl]-hexanamide,
N-(1-benzoyl-1,2,3,4-tetrahydro-2-methyl-4-quinolinyl)-N-(4chlorophenyl)-1,3-dioxo-1H-benz[de]isoquinoline-2(3H)-acetamide,
N-[1,1′-biphenyl]-3-yl-N-[1,2,3,4-tetrahydro-1-(4-methoxybenzoyl)-2-methyl-4-quinolinyl]acetamide,
N-(1-benzoyl-1,2,3,4-tetrahydro-2-methyl-4-quinolinyl)-N-(4-nitrophenyl)heptanamide,
N-(1-benzoyl-1,2,3,4-tetrahydro-2-methyl-4-quinolinyl)-N-(4-methoxyphenyl)-1,3-dioxo-1H-benz[de] isoquinoline-2(3H)-acetamide,
N-(1-benzoyl-1,2,3,4-tetrahydro-2-methyl-4-quinolinyl)-N-(4-methoxyphenyl)-2-methylpropanaminde,
N-[1-(4-fluorobenzoyl)-1,2,3,4-tetrahydro-2-methyl-4-quinolinyl]-N-phenylbutanamide,
2-phenoxy-N-phenyl-N-[1,2,3,4-tetrahydro-1-(2-methoxybenzoyl)-2-methyl-4-quinolinyl]acetamide,
N-phenyl-N-[1,2,3,4-tetrahydro-1-(3-methoxybenzoyl)-2-methyl-4-quinolinyl]pentanamide,
N-(2-methylphenyl)-2-(2-naphthalenyloxy)-N-[1,2,3,4-tetrahydro-2,8-dimethyl-1-(4-propylbenzoyl)-4-quinolinyl]acetamide,
N-phenyl-N-[1,2,3,4-tetrahydro-2-methyl-1-(4-nitrobenzoyl)-4-quinolinyl]octanamide,
N-[1-(4-ethylbenzoyl)-1,2,3,4-tetrahydro-2-methyl-4-quinolinyl]-2-(2-naphthalenyloxy)-N-phenylacetamide,
N-[1-(4-ethylbenzoyl)-1,2,3,4-tetrahydro-2,8-dimethyl-4-quinolinyl]-N-(2-methylphenyl)-3-(4-nitrophenyl)-2-propenamide,
N-(1-benzoyl-1,2,3,4-tetrahydro-2-methyl-4-quinolinyl)-2,2-dimethyl-N-phenylpropanamide,
N-(1-benzoyl-6-bromo-1,2,3,4-tetrahydro-2-methyl-4-quinolinyl)-N-phenylpentanamide,
2-methyl-N-phenyl-N-[1,2,3,4-tetrahydro-1-(3-methoxybenzoyl)-2-methyl-4-quinolinyl)propanamide,
2,2,2-trifluoro-N-phenyl-N-[1,2,3,4-tetrahydro-1-(3-methoxybenzoyl)-2-methyl-4-quinolinyl]acetamide,
N-[1-(4-ethylbenzoyl)-1,2,3,4-tetrahydro-2,8-dimethyl-4-quinolinyl]-N-(2-methylphenyl)-3-phenyl-2-propenamide,
N-(1-benzoyl-1,2,3,4-tetrahydro-2-methyl-4-quinolinyl)-N-(3-methoxyphenyl)acetamide,
N-[1-(4-chlorobenzoyl)-1,2,3,4-tetrahydro-2-methyl-4-quinolinyl]-1,3-dioxo-N-phenyl-1H-benz[de]isoquinoline-2(3H)-acetamide,
3-(4-nitrophenyl)-N-phenyl-N-[1,2,3,4-tetrahydro-2-methyl-1-(4-propylbenzoyl)-4-quinolinyl]-2-propenamide,
N,3-diphenyl-N-[1,2,3,4-tetrahydro-1-(4-methoxybenzoyl)-2-methyl-4-quinolinyl]-2-propenamide,
N-[1-(2-fluorobenzoyl)-1,2,3,4-tetrahydro-2-methyl-4-quinolinyl]-N-phenylhexanamide,
N-phenyl-N-[1,2,3,4-tetrahydro-1-(4-methoxybenzoyl)-2-methyl-4-quinolinyl]hexanamide,
N-(1-benzoyl-1,2,3,4-tetrahydro-2-methyl-4-quinolinyl)-N-(4-methylphenyl)acetamide,
3-(4-nitrophenyl)-N-phenyl-N-[1,2,3,4-tetrahydro-1-(4-methoxybenzoyl)-2-methyl-4-quinolinyl]-2-propenamide,
3-(4-methoxyphenyl)-N-phenyl-N-[1,2,3,4-tetrahydro-1-(4-methoxybenzoyl)-2-methyl-4-quinolinyl]-2-propenamide,
N-[1-(4-chloro-3-nitrobenzoyl)-1,2,3,4-tetrahydro-2-methyl-4-quinolinyl]-N-phenylacetamide,
1,3-dioxo-N-phenyl-N-[1,2,3,4-tetrahydro-1-(4-methoxybenzoyl)-2-methyl-4-quinolinyl]-1H-benz[de]isoquinoline-2(3H)-acetamide,
N-phenyl-N-[1,2,3,4-tetrahydro-2-methyl-1-(3-nitrobenzoyl)-4-quinolinyl]acetamide,
N-phenyl-N-[1,2,3,4-tetrahydro-1-(3-methoxypbenzoyl)-2-methyl-4-quinolinyl]hexanamide,
N-[1-(3-chlorobenzoyl)-1,2,3,4-tetrahydro-2-methyl-4-quinolinyl]-N-phenylacetamide,
N-[(2R,4S)-1-benzoyl-1,2,3,4-tetrahydro-2-methyl-4-quinolinyl]-2-methyl-N-phenylpropanamide,
N-[(2R,4S)-1-benzoyl-1,2,3,4-tetrahydro-2-methyl-4-quinolinyl]-2,2dimethyl-N-phenylpropanamide,
N-[1-(3-fluorobenzoyl)-1,2,3,4-tetrahydro-2-methyl-4-quinolinyl]-N-phenylacetamide,
N-[1-[4-(1,1-dimethylethyl)benzoyl]-1,2,3,4-tetrahydro-2-methyl-4-quinolinyl]-N-phenylacetamide,
rel-N-[(2R,4S)-1-benzoyl-1,2,3,4-tetrahydro-2-methyl-4-quinolinyl]-N-phenylbutanamide,
rel-N-[(2R,4S)-1-benzoyl-1,2,3,4-tetrahydro-2-methyl-4-quinolinyl]-N-phenylacetamide,
rel-N-[(2R,4S)-1-benzoyl-1,2,3,4-tetrahydro-2-methyl-4-quinolinyl]-N-phenylheptanamide,
rel-N-[(2R,4S)-1-benzoyl-1,2,3,4-tetrahydro-2-methyl-4-quinolinyl]-N-phenylpentanamide,
N-phenyl-N-[1,2,3,4-tetrahydro-1-(4-methoxybenzoyl)-2-methyl-4-quinolinyl]acetamide,
N-[1-(3,5-dinitrobenzoyl)-1,2,3,4-tetrahydro-2-methyl-4-quinolinyl]-N-phenylacetamide,
N-phenyl-N-[1,2,3,4-tetrahydro-2-methyl-1-(4-nitrobenzoyl)-4-quinolinyl]acetamide,
N-phenyl-N-[1,2,3,4-tetrahydro-1-(2-iodobenzoyl)-2-methyl-4-quinolinyl]acetamide,
N-phenyl-N-[1,2,3,4-tetrahydro-1-(3-methoxybenzoyl)-2-methyl-4-quinolinyl]acetamide,
1,3-dihydro-1,3-dioxo-N-phenyl-N-[1,2,3,4-tetrahydro-1-(3-methoxybenzoyl)-2-methyl-4-quinolinyl]-2H-isoinodole-2-acetamide,
N-(1-benzoyl-1,2,3,4-tetrahydro-2-methyl-4-quinolinyl)-N-phenylhexanamide,
N-(1-benzoyl-1,2,3,4-tetrahydro-2-methyl-4-quinolinyl)-N-phenylpentanamide,
N-(1-benzoyl-1,2,3,4-tetrahydro-2-methyl-4-quinolinyl)-N-phenylbutanamide,
N-(1-benzoyl-1,2,3,4-tetrahydro-2-methyl-4-quinolinyl)-N-phenylpropanamide,
1-benzoyl-1,2,3,4-tetrahydro-4-(N-phenylacetamido)quinaldine,
N-(1-benzoyl-6-chloro-1,2,3,4-tetrahydro-2-methyl-4-quinolinyl)acetanilide and
1-benzoyl-6-bromo-1,2,3,4-tetrahydro-4-(N-phenylacetamido)quinaldine.

Exemplary commercially available compounds of formula VII include:
1,2,3,4-tetrahydro-1-(3-iodobenzoyl)-2,6-dimethyl-4-phenylquinoline and
1-benzoyl-1,2,3,4-tetrahydro-4-(4-hydroxyphenyl)-2,6-dimethylquinoline.
   Preparation of the Compounds

Synthesis routes to the compounds provided herein are described in the Examples. One of skill in the art will understand that the synthetic routes can be modified to use different starting materials and/or alternate reagents to accomplish the desired transformations. Additionally, one of skill in the art will recognize that protecting groups may be necessary for the preparation of certain compounds and will be aware of those conditions compatible with a selected protecting group. Accordingly, the methods and reagents described herein are all expressed as non-limiting embodiments.

Compositions

In another aspect, the invention provides pharmaceutical compositions suitable for pharmaceutical use comprising one or more compounds of the invention and a pharmaceutically acceptable carrier, excipient or diluent.

The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant that the carrier or excipient is compatible with the other ingredients of the formulation and not deleterious to the recipient thereof.

Formulation may improve one or more pharmacokinetic properties (e.g., oral bioavailability, membrane permeability) of a compound of the invention (herein referred to as the active ingredient).

The pharmaceutical compositions for the administration of the compounds of this invention may conveniently be presented in unit dosage form and may be prepared by any of the methods well known in the art. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452 and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the invention are employed. As used herein, topical application is also meant to include the use of mouthwashes and gargles.

The pharmaceutical compositions and methods of the invention may further comprise other therapeutically active compounds, as noted herein, useful in the treatment of asthma, allergic and other immune-related diseases, inflammatory conditions and cancer and pathologies associated therewith (e.g., cardiovascular disease) or adjuvant(s). In many instances, compositions which include a compound of the invention and an alternative agent have additive or synergistic effects when administered.

Methods of Use

In yet another aspect, the invention provides methods of treating or preventing a disease or condition associated with CRTH2 and/or one or more other PGD₂ receptors by administering to a subject having such a condition or disease, a therapeutically effective amount of a compound or composition of the invention. In one group of embodiments, diseases and conditions, including chronic diseases of humans or other species, can be treated with modulators, or antagonists, of CRTH2 and/or one or more other PGD₂ receptors. These diseases and conditions include (1) inflammatory or allergic diseases such as systemic anaphylaxis and hypersensitivity disorders, atopic dermatitis, urticaria, drug allergies, insect sting allergies, food allergies (including celiac disease and the like) and mastocytosis, (2) inflammatory bowel diseases such as Crohn's disease, ulcerative colitis, ileitis and enteritis, (3) vasculitis, Behcet's syndrome, (4) psoriasis and inflammatory dermatoses such as dermatitis, eczema, atopic dermatitis, allergic contact dermatitis, urticaria, viral cutaneous pathologies such as those derived from human papillomavirus, HIV or RLV infection, bacterial, flugal and other parasital cutaneous pathologies, and cutaneous lupus erythematosus, (5) asthma and respiratory allergic diseases such as allergic asthma, allergic rhinitis, otitis media, allergic conjunctivitis, hypersensitivity lung diseases, chronic obstructive pulmonary disease and the like, (6) autoimmune diseases, such as arthritis (including rheumatoid and psoriatic), systemic lupus erythematosus, type I diabetes, myasthenia gravis, multiple sclerosis, Graves' disease, glomerulonephritis and the like, (7) graft rejection (including allograft rejection and graft-v-host disease), e.g., skin graft rejection, solid organ transplant rejection, bone marrow transplant rejection, (8) fever, (9) cardiovascular disorders such as acute heart failure, hypotension, hypertension, angina pectoris, myocardial infarction, cardiomyopathy, congestive heart failure, atherosclerosis, coronary artery disease, restenosis and vascular stenosis, (10) cerebrovascular disorders such as traumatic brain injury, stroke, ischemic reperfusion injury and aneurysm, (11) cancers of the breast, skin, prostate, cervix, uterus, ovary, testes, bladder, lung, liver, larynx, oral cavity, colon and gastrointestinal tract (e.g., esophagus, stomach, pancreas), brain, thyroid, blood and lymphatic system, (12) fibrosis, connective tissue disease and sarcoidosis, (13) genital and reproductive conditions such as erectile dysfunction, (14) gastrointestinal disorders such as gastritis, ulcers, nausea, pancreatitis and vomiting; (15) neurologic disorders, such as Alzheimer's disease, (16) sleep disorders such as insomnia, narcolepsy, sleep apnea syndrome and Pickwick Syndrome, (17) pain, (18) renal disorders, (19) ocular disorders such as glaucoma, and (20) infectious diseases such as HIV.

In yet another aspect, the invention provides methods of treating or preventing a disease or disorder responsive to modulation of CRTH2 and/or one or more other PGD₂ receptors comprising administering to a subject having such a disease or disorder, a therapeutically effective amount of one or more of the subject compounds or compositions.

In yet another aspect, the invention provides methods of treating or preventing a disease or disorder mediated by CRTH2 and/or one or more other PGD₂ receptors comprising administering to a subject having such a condition or disease, a therapeutically effective amount of one or more of the subject compounds or compositions.

In yet another aspect, the invention provides methods of modulating CRTH2 and/or one or more other PGD₂ receptors comprising contacting a cell with one or more of the subject compounds or compositions.

Depending on the disease to be treated and the subject's condition, the compounds of the invention may be administered by oral, parenteral (e.g., intramuscular, intraperitoneal, intravenous, ICV, intracistemal injection or infusion, subcutaneous injection or implant), inhalation, nasal, vaginal, rectal, sublingual, or topical (e.g., transdermal, local) routes of administration and may be formulated, alone or together, in suitable dosage unit formulations containing conventional non-toxic pharmaceutically acceptable carriers, adjuvants and vehicles appropriate for each route of administration. The invention also contemplates administration of the compounds of the invention in a depot formulation, in which the active ingredient is released over a defined time period.

In the treatment or prevention of asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease, cancer or other conditions or disorders associated with CRTH2 and/or one or more other PGD₂ receptors, an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the invention can be combined or used in combination with other agents useful in the treatment, prevention, suppression or amelioration of the diseases or conditions for which compounds of the invention are useful, including asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease, cancer and those pathologies noted above.

Such other agents, or drugs, may be administered, by a route and in an amount commonly used therefor, simultaneously or sequentially with a compound of the invention. When a compound of the invention is used contemporaneously with one or more other drugs, a pharmaceutical composition containing such other drugs in addition to the compound of the invention may be preferred. Accordingly, the pharmaceutical compositions of the invention include those that also contain one or more other active ingredients or therapeutic agents, in addition to a compound of the invention.

Examples of other therapeutic agents that may be combined with a compound of the invention, either administered separately or in the same pharmaceutical compositions, include, but are not limited to: (a) VLA-4 antagonists, (b) corticosteroids, such as beclomethasone, methylprednisolone, betamethasone, prednisone, prenisolone, dexamethasone, fluticasone and hydrocortisone, and corticosteroid analogs such as budesonide; (c) immunosuppressants such as cyclosporine (cyclosporine A, Sandimmune®, Neoral®) , tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astemizole, terfenadine, loratadine, cetirizine, fexofenadine, descarboethoxyloratadine and the like; (e) non-steroidal anti-asthmatics such as β2-agonists (e.g., terbutaline, metaproterenol, fenoterol, isoetharine, albuterol, bitolterol and pirbuterol) and β2-agonist-corticosteroid combinations (e.g., salmeterol-fluticasone (Advair®), formoterol-budesonid (Symbicort®)), theophylline, cromolyn sodium, atropine, ipratropium bromide, leukotriene antagonists (e.g., zafirlukast, montelukast, pranlukast, iralukast, pobilukast and SKB-106,203), leukotriene biosynthesis inhibitors (zileuton, BAY-1005); (f) non-steroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) other PGD₂ receptor antagonists, especially DP antagonists; (j) opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine; (k) cholesterol lowering agents such as HMG-CoA reductase inhibitors (e.g., lovastatin, simvastatin, pravastatin, fluvastatin, atorvastatin and other statins), bile acid sequestrants (e.g., cholestyramine and colestipol), vitamin B₃ (also known as nicotinic acid, or niacin), vitamin B₆ (pyridoxine), vitamin B₁₂ (cyanocobalamin), fibric acid derivatives (e.g., gemfibrozil, clofibrate, fenofibrate and benzafibrate), probucol, nitroglycerin, and inhibitors of cholesterol absorption (e.g., beta-sitosterol and acylCoA-cholesterol acyltransferase (ACAT) inhibitors such as melinamide), HMG-CoA synthase inhibitors, squalene epoxidase inhibitors and squalene synthetase inhibitors; (1) antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat); (m) anti-diabetic agents such as insulin and insulin mimetics, sulfonylureas (e.g., glyburide, meglinatide), biguanides, e.g., metformin (Glucophage®), α-glucosidase inhibitors (acarbose), thiazolidinone compounds, e.g., rosiglitazone (Avandia®), troglitazone (Rezulin®), ciglitazone, pioglitazone (Actos®) and englitazone; (n) preparations of interferon beta (interferon β-1 α, interferon β-1 β); (o) gold compounds such as auranofin and aurothioglucose, (p) TNF inhibitors, e.g., etanercept (Enbrel®), antibody therapies such as orthoclone (OKT3), daclizumab (Zenapax®), basiliximab (Simulect®), infliximab (Remicade®) and D2E6 TNF antibody, (q) lubricants or emollients such as petrolatum and lanolin, keratolytic agents, vitamin D₃ derivatives (e.g., calcipotriene and calcipotriol (Dovonex®)), PUVA, anthralin (Drithrocreme®), etretinate (Tegison®) and isotretinoin; (r) multiple sclerosis therapeutic agents such as interferon β-1β (Betaseron®), interferon β-1α (Avonex®), azathioprine (Imurek®, Imuran®), glatiramer acetate (Capoxone®), a glucocorticoid (e.g., prednisolone) and cyclophosphamide; (s) other compounds such as 5-aminosalicylic acid and prodrugs thereof; (t) DNA-alkylating agents (e.g., cyclophosphamide, ifosfamide), antimetabolites (e.g., azathioprine, 6-mercaptopurine, methotrexate, a folate antagonist, and 5-fluorouracil, a pyrimidine antagonist), microtubule disruptors (e.g., vincristine, vinblastine, paclitaxel, colchicine, nocodazole and vinorelbine), DNA intercalators (e.g., doxorubicin, daunomycin and cisplatin), DNA synthesis inhibitors such as hydroxyurea, DNA cross-linking agents, e.g., mitomycin C, hormone therapy (e.g., tamoxifen and flutamide), and cytostatic agents, e.g., imatinib (ST1571, Gleevec®) and rituximab (Rituxan®). The weight ratio of the compound of the invention to the second active ingredient may be varied and will depend upon the effective dose of each ingredient. Generally, an effective dose of each will be used. Thus, for example, when a compound of the invention is combined with an NSAID, the weight ratio of the compound of the invention to the NSAID will generally range from about 1000:1 to about 1:1000, preferably about 200:1 to about 1:200. Combinations of a compound of the invention and other active ingredients will generally also be within the aforementioned range, but in each case, an effective dose of each active ingredient should be used.

Analysis of the Compounds

In yet another aspect, the invention includes methods to evaluate putative specific agonists or antagonists of CRTH2 and/or one or more other PGD₂ receptors. Accordingly, the invention is directed to the use of these compounds in the preparation and execution of screening assays for compounds which modulate the function of CRTH2 and/or one or more other PGD₂ receptors. For example, the compounds of this invention are useful for CRTH2 mutants and/or one or more other PGD₂ receptor mutants, which are excellent screening tools for potent compounds. Furthermore, the compounds of this invention are useful in establishing or determining the binding site of other compounds to CRTH2 and/or one or more other PGD₂ receptors, e.g., by competitive inhibition. The compounds of the instant invention are also useful for the evaluation of putative specific modulators of CRTH2 and/or one or more other PGD₂ receptors. One of skill in the art will appreciate that thorough evaluation of specific agonists and antagonists of PGD₂ receptors has been hampered by the lack of availability of non-peptidyl (metabolically resistant) compounds with high binding affinity for these receptors. The compounds provided herein are particularly useful in this context.

High Throughput Screening

High throughput assays for the presence, absence, quantification, or other properties of particular compounds may be used to test a combinatorial library that contains a large number of potential therapeutic compounds (potential modulator compounds). The assays are typically designed to screen large chemical libraries by automating the assay steps and providing compounds from any convenient source to the assays, which are typically run in parallel (e.g., in microtiter formats on microtiter plates in robotic assays). Preferred assays detect enhancement or inhibition of CRTH2 and/or one or more other PGD₂ receptors function.

High throughput screening systems are commercially available (see e.g., Zymark Corp., Hopkinton Mass.; Air Technical Industries, Mentor Ohio; Beckman Instruments, Inc., Fullerton Calif.; Precision Systems, Inc., Natick Mass.; etc.). These systems typically automate entire procedures, including all sample and reagent pipetting, liquid dispensing, timed incubations, and final readings of the microplate in detector(s) appropriate for the assay. These configurable systems provide high throughput and rapid start-up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

The following examples are offered by way of illustration and are not intended to limit the scope of the invention. Those of skill in the art will readily recognize a variety of noncritical parameters that could be modified to yield essentially similar results.

### EXAMPLES

Reagents and solvents used below can be obtained from commercial sources such as Aldrich Chemical Co. (Milwaukee, Wis., USA). ¹H-NMR spectra were recorded on a Varian Gemini 400 MHz NMR spectrometer. Significant peaks are tabulated in the order: multiplicity (s, singlet; d, doublet; t, triplet; q, quartet; m, multiplet; br s, broad singlet), number of protons and coupling constant(s) in Hertz (Hz). Mass spectrometry results are reported as the ratio of mass over charge, followed by the relative abundance of each ion (in parentheses) or a single m/z value for the M+H (or, as noted, M-H) ion containing the most common atomic isotopes. Isotope patterns correspond to the expected formula in all cases. Electrospray ionization (ESI) mass spectrometry analysis was conducted on a Hewlett-Packard 1100 MSD electrospray mass spectrometer using the HP1100 HPLC for sample delivery. Normally the analyte was dissolved in methanol at 0.1 mg/mL and 1 microliter was infused with the delivery solvent into the mass spectrometer, which scanned from 100 to 1500 daltons. All compounds could be analyzed in the positive ESI mode, using 1:1 acetonitrile/water with 1% formic acid as the delivery solvent. The compounds provided below could also be analyzed in the negative ESI mode, using 5 mM NH₄OAc in acetonitrile/water as delivery solvent.

### Example 1

This example illustrates the preparation of compound 4 from aniline and acetaldehyde.

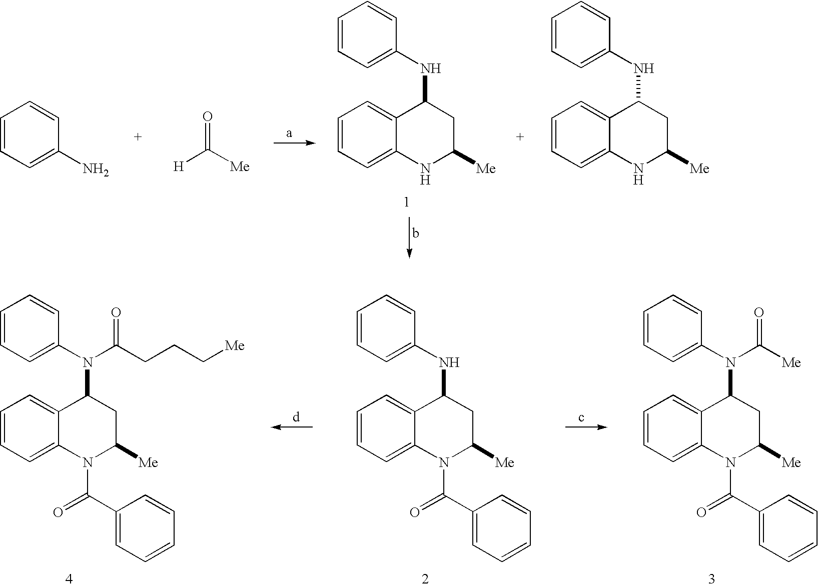

Step a. Treatment with ethanol at r.t. for 3 days according to the procedure described in Funabashi et al. (1969) Bull. Chem. Soc. Jpn. 42:2885-2894 afforded a mixture of compound 1 and the trans isomer. Compound 1: ¹H NMR (CDCl₃) δ 7.38 (d, 1H, J=7.7 Hz), 7.20 (m, 2H), 7.04 (m, 1H), 6.70 (m, 4H), 6.51 (d, 1H, J=8.0 Hz), 4.83 (m, 1H), 3.85 (br s, 2H), 3.64 (m, 1H), 2.38 (m, 1H), 1.52 (t, 1H, J=12.4 Hz), 1.23 (d, 3H, J=6.2 Hz). MS (ESI⁺) 239.1 [MH]⁺.

Step b. Benzoyl chloride (0.36 mL, 3.12 mmol) was added to a mixture of 1 (675 mg, 2.84 mmol) and triethylamine (0.44 mL, 3.12 mmol) in dichloromethane (5 mL) at 0° C. The mixture was stirred at r.t. for 2 days and treated with dichloromethane (50 mL) and saturated aqueous sodium bicarbonate (50 mL). The organic layer was separated, dried with sodium sulfate and concentrated. The residue was washed with ether to give compound 2. ¹H NMR (CDCl₃) δ 7.40-7.20 (m, 8H), 7.07 (t, 1H), 6.91 (t, 1H), 6.79 (t, 1H), 6.70 (d, 2H), 6.53 (d, 1H), 4.94 (m, 1H), 4.48 (m, 1H), 3.89 (d, 1H), 2.84 (m, 1H), 1.42 (m, 1H), 1.29 (d, 3H). MS (ESI⁺) 343.2 [MH]⁺.

Step c. Sodium hydride (7.6 mg, 0.3 mmol) was added to a solution of 2 (34 mg, 0.1 mmol) in THF (1 mL) at 0° C., followed by acetyl bromide (0.022 mL, 0.3 mmol). The mixture was stirred at r.t. for 12 h and treated with dichloromethane (5 mL) and saturated aqueous sodium bicarbonate (5 mL). The organic layer was separated, dried with sodium sulfate and concentrated. The residue was purified by column (eluting with 30% EtOAc/Hexanes) to afford compound 3. ¹H NMR (CDCl₃) δ 7.45-7.10 (m, 12H), 6.88 (t, 1H), 6.49 (d, 1H), 5.64 (br s, 1H), 4.80 (m, 1H), 2.34 (m, 1H), 2.04 (s, 3H), 1.22 (br s, 1H), 1.15 (d, 3H). MS (ESI⁺) 385.2 [MH]⁺.

Step d. Compound 4 was synthesized according to the above-described procedure (step c) using valeryl chloride. ¹H NMR (CDCl₃) δ 7.45-7.10 (m, 12H), 6.88 (t, 1H), 6.49 (d, 1H), 5.64 (br s, 1H), 4.80 (m, 1H), 2.40-2.10 (m, 3H), 1.66 (m, 2H), 1.30 (m, 2H), 1.16 (d, 3H), 0.91 (m, 1H), 0.86 (t, 3H). MS (ESI⁺) 426.2 [MH]⁺.

### Example 2

This example illustrates the preparation of compound 9 from aniline and ethyl acetoacetate.

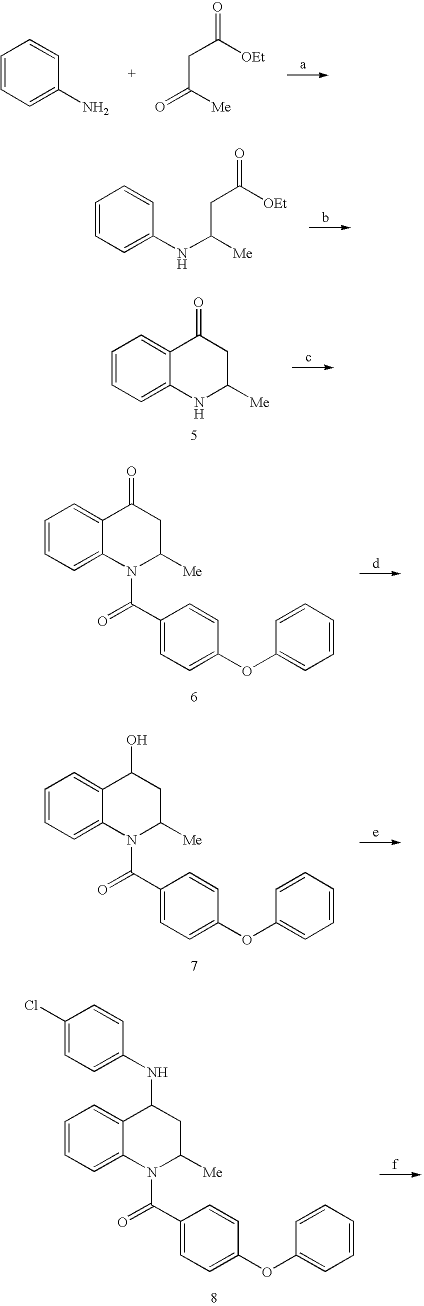

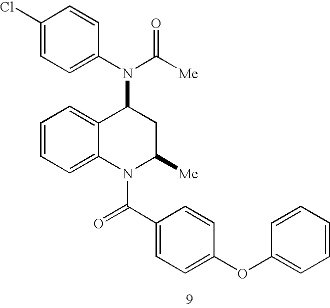

Step a. Sodium triacetoxyborohydride (32 g, 150 mmol) was added to a mixture of aniline (9.1 mL), ethyl acetylacetate (12.7 mL, 100 mmol), and acetic acid (7.4 mL, 130 mmol) in 1,2-dichloroethane at r.t. The mixture was stirred at r.t. for 16 h and treated with dichloromethane (500 mL) and saturated aqueous sodium carbonate (500 mL). The organic layer was separated, dried with sodium sulfate and concentrated. The residue was used in the next step without further purification.

Step b. A mixture of the product of step a (3 g) and PPA (41 g) was heated to 110° C. with stirring for 14 h. After cooling, the mixture was treated with ice water/dichloromethane and neutralized with saturated sodium carbonate. The organic layer was separated, dried with sodium sulfate and concentrated to give compound 5 which was purified by column chromatography (eluting with 20% EtOAc/Hexanes). ¹H NMR (CDCl₃) δ 7.83 (d, 1H), 7.30 (t, 1H), 6.74 (t, 1H), 6.66 (d, 1H), 4.30 (br s, 1H), 3.79 (m, 1H), 2.63 (m, 1H), 2.48 (m, 1H), 1.34 (d, 3H). MS (ESI⁺) 162.1 [MH]⁺.

Step c. Compound 6 was synthesized according to the procedure described in Example 1, step b using 4-phenoxybenzoyl chloride.

Step d. Sodium borohydride (24 mg, 0.62 mmol) was added to a solution of 6 (220 mg, 0.62 mmol) in methanol (2 mL). The mixture was stirred at r.t. for 1 h and treated with dichloromethane (20 mL) and saturated aqueous sodium chloride (20 mL). The organic layer was separated, dried with sodium sulfate and concentrated. The residue was used in the next step without further purification.

Step e. Trimethylsilyl iodide (0.057 mL, 0.4 mmol) was added to a solution of the product of step d above (7) in dichloromethane at 0° C. The mixture was stirred at 0° C. for 4 h and concentrated. The residue was dissolved in THF and treated with barium carbonate (30 mg, 0.15 mmol) and 4-chloroaniline (19 mg, 0.15 mmol). The reaction mixture was stirred at r.t. for 16 h and treated with dichloromethane (20 mL) and saturated aqueous sodium bicarbonate (20 mL). The organic layer was separated, dried with sodium sulfate and concentrated. The residue was used in the next step without further purification.

Step f. Sodium hydride (13 mg, 0.5 mmol) was added to the product of step e above (8, 0.1 mmol) in THF (2 mL) at 0° C., followed by acetyl chloride(0.029 mL, 0.4 mmol). The mixture was stirred at 60° C. for 14 h and treated with dichloromethane (5 mL) and saturated aqueous sodium bicarbonate (5 mL). The organic layer was separated, dried with sodium sulfate and concentrated. The residue contained both cis and trans isomers, which were separated by column (eluting with 30% EtOAc/Hexanes) to afford compound 9. ¹H NMR (CDCl₃) δ 7.50-7.10 (m, 11H), 7.05 (m, 3H), 6.84 (m, 2H), 6.62 (d, 1H), 5.64 (br s, 1H), 4.85 (m, 1H), 2.38 (m, 1H), 2.17 (s, 3H), 1.25 (br s, 1H), 1.23 (d, 3H). MS (ESI⁺) 511.2 [MH]⁺.

### Example 3

This example illustrates the preparation of compound 12 from 4-oxo-tetrahydroquinoline 5.

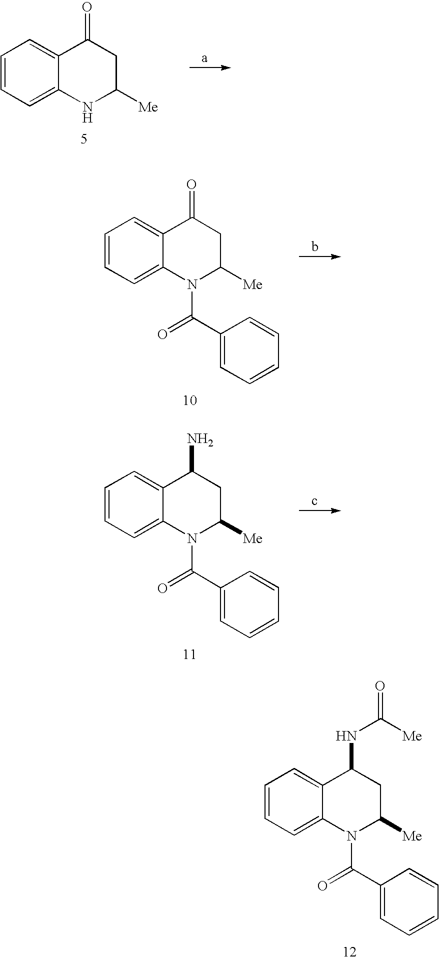

Step a. Compound 10 was synthesized according to the procedure described in Example 1, step b using benzoyl chloride. ¹H NMR (CDCl₃) δ 8.01 (dd, 1H), 7.49 (m, 2H), 7.44 (m, 1H), 7.34 (m, 2H), 7.23 (m, 1H), 7.13 (m, 1H), 6.78 (d, 1H), 5.28 (m, 1H), 3.14 (dd, 1H), 2.68 (dd, 1H), 1.33 (d, 3H). MS (ESI⁺) 266.1 [MH]⁺.

Step b. Treatment with NH₄OAc and NaBH₃CN in methanol at r.t. for 2 days. Sodium cyanoborohydride (63 mg, 1 mmol) was added to a mixture of 10 (53 mg, 0.2 mmol) and ammonium acetate (154 mg, 2 mmol) in methanol (3 mL). The mixture was stirred at 70° C. for 2 days and diluted with dichloromethane (20 mL) and saturated aqueous sodium bicarbonate (20 mL). The organic layer was separated, dried with sodium sulfate and concentrated. The residue was used in the next step without further purification.

Step c. Acetyl bromide (0.0056 mL, 0.075 mmol) was added to the product of step b above (11, 13 mg, 0.05 mmol) and triethylamine (0.014 mL, 0.1 mmol) in dichloromethane. The mixture was stirred at r.t. for 14 h and treated with dichloromethane (5 mL) and saturated aqueous sodium bicarbonate (5 mL). The organic layer was separated, dried with sodium sulfate and concentrated. The residue was purified by column chromatography (eluting with 70% EtOAc/Hexanes) to afford compound 12. ¹H NMR (CDCl₃) δ 7.30-7.12 (m, 6H), 7.08 (t, 1H), 6.90 (t, 1H), 6.52 (d, 1H), 5.68 (d, 1H), 5.19 (m, 1H), 4.88 (m, 1H), 2.70 (m, 1H), 2.17 (s, 3H), 1.40 (m, 1H), 1.27 (d, 3H). MS (ESI⁺) 309.2 [MH]⁺.

### Example 4

This example illustrates the preparation of compound 14 from 4-oxo-tetrahydroquinoline 10.

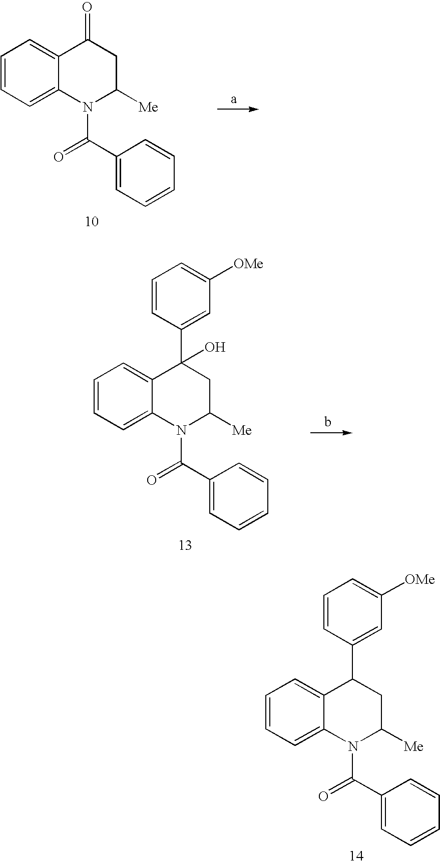

Step a. 3-Methoxyphenyl magnesium bromide (0.6 mL, 1.0 M in THF, 0.6 mmol) was added to a solution of 10 (50 mg, 0.2 mmol) in THF (1 mL). The mixture was stirred at r.t. for 16 h and treated with dichloromethane (5 mL) and saturated aqueous sodium bicarbonate (5 mL). The organic layer was separated, dried with sodium sulfate and concentrated. The residue was purified by column chromatography (eluting with 40% EtOAc/Hexanes) to give compound 13. ¹H NMR (CDCl₃) δ 7.71 (d, 1H), 7.30-7.15 (m, 3H), 7.06 (m, 4H), 6.95 (t, 1H), 6.79 (d, 3H), 6.54 (d, 1H), 4.86 (m, 1H), 3.76 (s, 3H), 3.15 (m, 1H), 2.25 (br s, 1H), 1.98 (m, 1H), 1.35 (d, 3H). MS (ESI⁺) 374.2 [MH]⁺.

Step b. A solution of 13 (23 mg) in TFA (0.5 mL) and dichloromethane (1 mL) was stirred at r.t. for 16 h. The mixture was concentrated under vacuum and the residue purified by column (eluting with 40% EtOAc/Hexanes) to afford compound 14.

¹H NMR (CDCl₃) δ 7.50-7.27 (m, 6H), 7.14 (d, 1H), 7.01 (m, 2H), 6.95 (m, 3H), 6.70 (br s, 1H), 6.16 (d, 1H), 5.40 (br s, 1H), 3.86 (s, 3H), 1.25 (d, 3H). MS (ESI⁺) 356.2 [MH]⁺.

### Example 5

This example describes an assay that may be used to identify compounds that modulate CRTH2 and/or one or more other PGD₂ receptors, e.g., DP receptor.

### Human CRTH2 Binding Assay

Full-length human CRTH2 cDNA was generated by polymerase chain reaction (PCR) using human genomic DNA as template and subsequently cloned into pcDNA3.1(+) (Invitrogen), generating a CRTH2 expression plasmid pHLT124. The plasmid was transfected into 293 cells, which normally express CRTH2, using LipofectAMINETM reagents (Gibco/BRL). G418 (800 mg/mL) was added to the culture 48 h after transfection and cells were maintained under selection for 3 weeks to ensure that all surviving cells stably expressed CRTH2. These cells are labeled as 293(124) hereafter.

³H-PGD₂ binding assay was performed using 293(124) cells. In brief, cells were washed and suspended in RPMI containing 0.5% BSA and 20 mM HEPES. Each assay contained 25,000 cells, appropriate amount of test compound when necessary and a mixture of 1 nM ³H-PGD₂ (Amersham Pharmacia Biotech) and 30 nM of unlabeled PGD₂ (Cayman Chemicals) in 200 mL final volume. The cell mixture was incubated at room temperature for 2.5 h with shaking and the cells were separated from free ³H-PGD₂ and transferred onto a filter plate using a cell harvester. Radioactivity bound to the cells was measured on a liquid scintillation counter. Nonspecific binding was determined in the presence of 10 mM of unlabeled PGD₂.

The above-described assay may be modified for use with another PGD₂ receptor (e.g., DP).

Compounds of the invention assessed by the above-described assay were found to modulate human CRTH2. See Table 1 below.

TABLE 1

| Compound | IC₅₀ (μM)¹ |
|---|---|
| 3 | ++ |
| 4 | + |
| 9 | + |
| 12 | + |
| 16 | ++ |
| 19 | ++ |
| 21 | + |

¹+ represents an IC₅₀ value of greater than or equal to 0.04 μM
++ represents an IC₅₀ value of less than 0.04 μM

Other Useful Assays

Modulation of CRTH2 and/or one or more other PGD₂ receptors by test compounds can be assessed by other in vitro and in vivo assays. Examples of such assays include measuring second messenger (e.g., cAMP, IP₃ or Ca²⁺) levels, ion flux, phosphorylation levels, transcription levels, and the like. Recombinant or naturally occurring CRTH2 polypeptides and/or other PGD₂ receptor peptides can be used and the protein can be isolated, expressed in a cell, expressed in a membrane derived from a cell, expressed in tissue or in an animal. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and/or cytoplasmic domain of a receptor. Gene amplification can also be examined. Furthermore, ligand-binding domains of the protein of interest can be used in vitro in soluble or solid state reactions to assay for ligand binding.

CRTH2-G-protein or another PGD₂ receptor-G-protein interactions can also be examined, by, for example, analysis of binding of the G-protein to the receptor or its release from the receptor.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound having the formula (I): 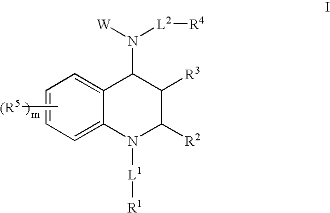
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(₂)═NR″, —S(O)R′, —SO₂R′, —SO₂NR′R″, —N₃ and —CH(Ph)₂;
optionally, two adjacent R⁵ groups may be combined to form a 5-, 6-, 7- or 8-membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4;
with the proviso that said compound is other than
N-(1-benzoyl-1,2,3,4-tetrahydro-2-methyl-4-quinolinyl)-2-methyl-N-phenylpropanamide,
N-[1-(3-fluorobenzoyl)-1,2,3,4-tetrahydro-2-methyl-4-quinolinyl]-N-phenylhexanamide,
N-[1-(4-ethylbenzoyl)-1,2,3,4-tetrahydro-2,8-dimethyl-4-quinolinyl]-N-(2-methylphenyl)-2-(2-naphthalenyloxy)acetamide,
N-phenyl-N-[1,2,3,4-tetrahydro-2-methyl-1-(3-nitrobenzoyl)-4-quinolinyl]-hexanamide,
N-(1-benzoyl-1,2,3,4-tetrahydro-2-methyl-4-quinolinyl)-N-(4-chlorophenyl)-1,3-dioxo-1H-benz[de]isoquinoline-2(3H)-acetamide,
N-[1,1′-biphenyl]-3-yl-N-[1,2,3,4-tetrahydro-1-(4-methoxybenzoyl)-2-methyl-4-quinolinyl]acetamide,
N-(1-benzoyl-1,2,3,4-tetrahydro-2-methyl-4-quinolinyl)-N-(4-nitrophenyl)heptanamide,
N-(1-benzoyl-1,2,3,4-tetrahydro-2-methyl-4-quinolinyl)-N-(4-methoxyphenyl)-1,3-dioxo-1H-benz[de]isoquinoline-2(3H)-acetamide,
N-(1-benzoyl-1,2,3,4-tetrahydro-2-methyl-4-quinolinyl)-N-(4-methoxyphenyl)-2-methylpropanamide,
N-[1-(4-fluorobenzoyl)-1,2,3,4-tetrahydro-2-methyl-4-quinolinyl]-N-phenylbutanamide, 2-phenoxy-N-phenyl-N-[1,2,3,4-tetrahydro-1-(2-methoxybenzoyl)-2-methyl-4-quinolinyl]acetamide,
N-phenyl-N-[1,2,3,4-tetrahydro-1-(3-methoxybenzoyl)-2-methyl-4-quinolinyl]pentanamide,
N-(2-methylphenyl)-2-(2-naphthalenyloxy)-N-[1,2,3,4-tetrahydro-2,8-dimethyl-1-(4-propylbenzoyl)-4-quinolinyl]acetamide,
N-phenyl-N-[1,2,3,4-tetrahydro-2-methyl-1-(4-nitrobenzoyl)-4-quinolinyl]octanamide,
N-[1-(4-ethylbenzoyl)-1,2,3,4-tetrahydro-2-methyl-4-quinolinyl]-2-(2-naphthalenyloxy)-N-phenylacetamide,
N-[1-(4-ethylbenzoyl)-1,2,3,4-tetrahydro-2,8-dimethyl-4-quinolinyl]-N-(2-methylphenyl)-3-(4-nitrophenyl)-2-propenamide,
N-(1-benzoyl-1,2,3,4-tetrahydro-2-methyl-4-quinolinyl)-2,2-dimethyl-N-phenylpropanamide,
N-(1-benzoyl-6-bromo-1,2,3,4-tetrahydro-2-methyl-4-quinolinyl)-N-phenylpentanamide,
2-methyl-N-phenyl-N-[1,2,3,4-tetrahydro-1-(3-methoxybenzoyl)-2-methyl-4-quinolinyl)propanamide,
2,2,2-trifluoro-N-phenyl-N-[1,2,3,4-tetrahydro-1-(3-methoxybenzoyl)-2-methyl-4-quinolinyl]acetamide,
N-[1-(4-ethylbenzoyl)-1,2,3,4-tetrahydro-2,8-dimethyl-4-quinolinyl]-N-(2-methylphenyl)-3-phenyl-2-propenamide,
N-(1-benzoyl-1,2,3,4-tetrahydro-2-methyl-4-quinolinyl)-N-(3methoxyphenyl)acetamide,
N-[1-(4-chlorobenzoyl)-1,2,3,4-tetrahydro-2-methyl-4-quinolinyl]-1,3-dioxo-N-phenyl-1H-benz[de]isoquinoline-2(3H)-acetamide,
3-(4-nitrophenyl)-N-phenyl-N-[1,2,3,4-tetrahydro-2-methyl-1-(4-propylbenzoyl)-4-quinolinyl]-2-propenamide,
N,3-diphenyl-N-[1,2,3,4-tetrahydro-1-(4-methoxybenzoyl)-2-methyl-4-quinolinyl]-2-propenamide,
N-[1-(2-fluorobenzoyl)-1,2,3,4-tetrahydro-2-methyl-4-quinolinyl]-N-phenylhexanamide,
N-phenyl-N-[1,2,3,4-tetrahydro-1-(4-methoxybenzoyl)-2-methyl-4-quinolinyl]hexanamide,
N-(1-benzoyl-1,2,3,4-tetrahydro-2-methyl-4-quinolinyl)-N-(4-methylphenyl)acetamide,
3-(4-nitrophenyl)-N-phenyl-N-[1,2,3,4-tetrahydro-1-(4-methoxybenzoyl)-2-methyl-4-quinolinyl]-2-propenamide,
3-(4-methoxyphenyl)-N-phenyl-N-[1,2,3,4-tetrahydro-1-(4-methoxybenzoyl)-2-methyl-4-quinolinyl]-2-propenamide,
N-[1-(4-chloro-3-nitrobenzoyl)-1,2,3,4-tetrahydro-2-methyl-4-quinolinyl]-N-phenylacetamide,
1,3-dioxo-N-phenyl-N-[1,2,3,4-tetrahydro-1-(4-methoxybenzoyl)-2-methyl-4-quinolinyl]-1H-benz[de]isoquinoline-2(3H)-acetamide,
N-phenyl-N-[1,2,3,4-tetrahydro-2-methyl-1-(3-nitrobenzoyl)-4-quinolinyl]acetamide,
N-phenyl-N-[1,2,3,4-tetrahydro-1-(3-methoxypbenzoyl)-2-methyl-4-quinolinyl]hexanamide,
N-[1-(3-chlorobenzoyl)-1,2,3,4-tetrahydro-2-methyl-4-quinolinyl]-N-phenylacetamide,
N-[(2R,4S)-1-benzoyl-1,2,3,4-tetrahydro-2-methyl-4-quinolinyl]-2-methyl-N-Response phenylpropanamide,
N-[(2R,4S)-1-benzoyl-1,2,3,4-tetrahydro-2-methyl-4-quinolinyl]-2,2-dimethyl-N-phenylpropanamide,
N-[1-(3-fluorobenzoyl)-1,2,3,4-tetrahydro-2-methyl-4-quinolinyl]-N-phenylacetamide,
N-[1-[4-(1,1-dimethylethyl)benzoyl]-1,2,3,4-tetrahydro-2-methyl-4-quinolinyl]-N-phenylacetamide,
rel-N-[(2R,4S)-1-benzoyl-1,2,3,4-tetrahydro-2-methyl-4-quinolinyl]-N-phenylbutanamide,
rel-N-[(2R,4S)-1-benzoyl-1,2,3,4-tetrahydro-2-methyl-4-quinolinyl]-N-phenylacetamide,
rel-N-[(2R,4S)-1-benzoyl-1,2,3,4-tetrahydro-2-methyl-4-quinolinyl]-N-phenylheptanamide,
rel-N-[(2R,4S)-1-benzoyl-1,2,3,4-tetrahydro-2-methyl-4-quinolinyl]-N-phenylpentanamide,
N-phenyl-N-[1,2,3,4-tetrahydro-1-(4-methoxybenzoyl)-2-methyl-4-quinolinyl]acetamide,
N-[1-(3,5-dinitrobenzoyl)-1,2,3,4-tetrahydro-2-methyl-4-quinolinyl]-N-phenylacetamide,
N-phenyl-N-[1,2,3,4-tetrahydro-2-methyl-1-(4-nitrobenzoyl)-4-quinolinyl]acetamide,
N-phenyl-N-[1,2,3,4-tetrahydro-1-(2-iodobenzoyl)-2-methyl-4-quinolinyl]acetamide,
N-phenyl-N-[1,2,3,4-tetrahydro-1-(3-methoxybenzoyl)-2-methyl-4-quinolinyl]acetamide,
1,3-dihydro-1,3-dioxo-N-phenyl-N-[1,2,3,4-tetrahydro-1-(3-methoxybenzoyl)-2-methyl-4-quinolinyl]-2H-isoinodole-2-acetamide,
N-(1-benzoyl-1,2,3,4-tetrahydro-2-methyl-4-quinolinyl)-N-phenylhexanamide,
N-(1-benzoyl-1,2,3,4-tetrahydro-2-methyl-4-quinolinyl)-N-phenylpentanamide,
N-(1-benzoyl-1,2,3,4-tetrahydro-2-methyl-4-quinolinyl)-N-phenylbutanamide,
N-(1-benzoyl-1,2,3,4-tetrahydro-2-methyl-4-quinolinyl)-N-phenylpropanamide,
1-benzoyl-1,2,3,4-tetrahydro-4-(N-phenylacetamido)quinaldine,
N-(1-benzoyl-6-chloro-1,2,3,4-tetrahydro-2-methyl-4-quinolinyl)acetanilide and
1-benzoyl-6-bromo-1,2,3,4-tetrahydro-4-(N-phenylacetamido)quinaldine.

2. The compound of claim 1, wherein L² is other then C(O) when R⁴ is unsubstituted alkyl.

3. The compound of claim 2, wherein R⁴ is selected from the group consisting of substituted (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl.

4. The compound of claim 3, wherein R⁴ is selected from the group consisting of aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl.

5. The compound of claim 1, wherein W is selected from the group consisting of phenyl, naphthyl, biphenyl, pyrrolyl, pyrazolyl, imidazolyl, pyrazinyl, oxazolyl, isoxazolyl, thiazolyl, furyl, thienyl, pyridyl, pyrimidyl, pyrimidinyl, pyridazinyl, benzothiazolyl, purinyl, benzimidazolyl, indolyl, indazolyl, carbazolyl, carbolinyl, isoquinolyl, quinoxalinyl and quinolyl.

6. The compound of claim 1, wherein L² is C(O).

7. The compound of claim 6, wherein W is phenyl.

8. The compound of claim 7, having the formula (II): 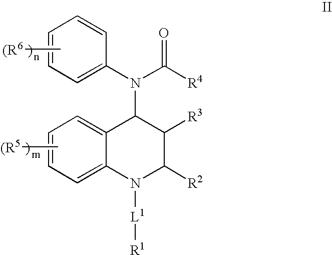
wherein
each R⁶ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —S(O)R′, —SO₂R′ and —SO₂NR′R″;
optionally, two adjacent R⁶ groups may be combined to form a 5-, 6-, 7- or 8-membered fused ring containing the carbon atoms to which they are attached and 0, 1 or 2 additional heteroatoms selected from N, O and S; and the subscript n is 0, 1, 2, 3, 4 or 5.

9. The compound of claim 1, wherein R¹ is selected from the group consisting of (C₁-C₈)alkyl, phenyl, naphthyl, aryl(C₁-C₄)alkyl, aryl(C₁-C₄)alkoxy, aryl(C₁-C₄)alkenyl or heteroaryl.

10. The compound of claim 9, wherein R¹ is unsubstituted phenyl or phenyl substituted with 1, 2 or 3 substituents selected from halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

11. The compound of claim 1, wherein L¹ is C(O).

12. The compound of claim 10, wherein R¹ is unsubstituted phenyl or phenyl substituted with 1, 2 or 3 substituents selected from the group consisting of halogen, (C₁-C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy.

13. The compound of claim 11, having the formula (III): 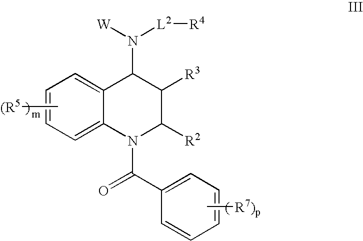
wherein
each R⁷ is independently selected from the group consisting of halogen, (C₁-C₈)alkyl, (C₁-C₈)alkoxy, halo(C₁-C₄)alkyl, halo(C₁-C₄)alkoxy, amino, (C₁-C₄)alkyl amino, di(C₁-C₄)alkylamino, nitro, cyano, aryl and aryloxy;
optionally, two adjacent R⁷ groups may be combined to form a 5-, 6-, 7- or 8-membered fused ring containing the carbon atoms to which they are attached and 0, 1 or 2 additional heteroatoms selected from N, O and S; and
the subscript p is 0, 1, 2, 3, 4 or S.

14. The compound of claim 13, wherein the subscript p is 1.

15. The compound of claim 14, having the formula (IV):

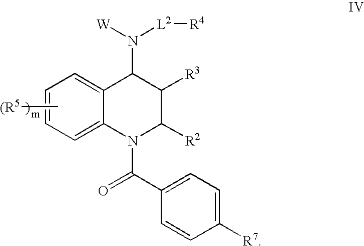

16. The compound of claim 1, wherein R³ is hydrogen.

17. The compound of claim 1, having the formula (V): 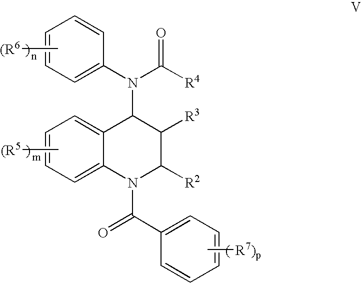
wherein
each R⁶ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —S(O)R′, —SO₂R′ and —SO₂NR′R″;
optionally, two adjacent R⁶ groups may be combined to form a 5-, 6-, 7- or 8-membered fused ring containing the carbon atoms to which they are attached and 0, 1 or 2 additional heteroatoms selected from N, O and S;
each R⁷ is independently selected from the group consisting of halogen, (C₁-Response C₈)alkyl, (C₁-C₈)alkoxy, halo(C₁-C₄)alkyl, halo(C₁-C₄)alkoxy, amino, (C₁-C₄)alkylamino, di(C₁-C₄)alkylamino, nitro, cyano, aryl and aryloxy;
optionally, two adjacent R⁷ groups may be combined to form a 5-, 6-, 7- or 8-membered fused ring containing the carbon atoms to which they are attached and 0, 1 or 2 additional heteroatoms selected from N, O and S; and
the subscripts n and p are independently 0, 1, 2, 3, 4 or 5.

18. A pharmaceutical composition comprising a pharmaceutically acceptable carrier or excipient and a compound of formula (I): 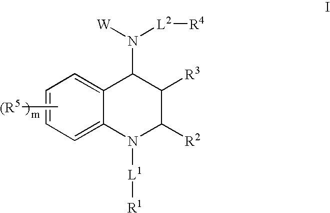
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ and —CH(Ph)₂;
optionally, two adjacent R⁵ groups may be combined to form a 5-, 6-, 7- or 8-membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4.

19. A pharmaceutical composition comprising a pharmaceutically acceptable carrier or excipient and a compound of any one of claims 1-17.

20. A method for treating a disease or condition selected from the group consisting of asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease and cancer, comprising administering to a subject in need thereof a therapeutically effective amount of a compound of formula (I): 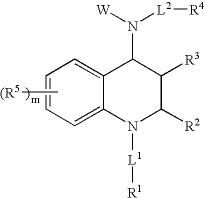
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ and —CH(Ph)₂;
optionally, two adjacent R⁵ groups may be combined to form a 5-, 6-, 7- or 8-membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4.

21. A method for treating a disease or condition selected from the group consisting of asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease and cancer, comprising administering to a subject in need thereof a therapeutically effective amount of a compound of any one of claims 1-17.

22. A method for treating a disease or condition responsive to the modulation of CRTH2 and/or one or more other PGD₂ receptors, comprising administering to a subject in need thereof a therapeutically effective amount of a compound of formula (I): 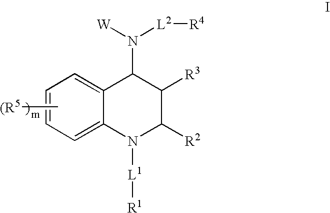
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ and —CH(Ph)₂;
optionally, two adjacent R⁵ groups may be combined to form a 5-, 6-, 7- or 8-membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4.

23. (Canceled)

24. The method of claim 22, wherein said disease or condition is selected from the group consisting of asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease and cancer.

25. The method of claim 24, wherein said compound is administered orally, parenterally or topically.

26. The method of claim 25, wherein said compound is administered in combination with a second therapeutic agent.

27. The method of claim 26, wherein said second therapeutic agent is useful for treating asthma, allergic rhinitis, eczema, psoriasis, atopic dermatitis, fever, sepsis, systemic lupus erythematosus, diabetes, rheumatoid arthritis, multiple sclerosis, atherosclerosis, transplant rejection, inflammatory bowel disease or cancer.

28. A method for modulating the function of CRTH2 and/or one or more other PGD₂ receptors in a cell, comprising contacting a cell with a compound of formula (I): 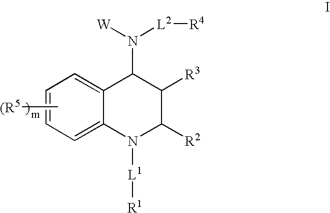
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, SO₂R′, —SO₂NR′R″, —N₃ and —CH(Ph)₂;
optionally, two adjacent R⁵ groups may be combined to form a 5-, 6-, 7- or 8-membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4.

29. (Canceled)

30. A method for modulating CRTH2 and/or one or more other PGD₂ receptors, comprising contacting a CRTH2 protein and/or one or more other PGD₂ receptors proteins with a compound of formula (I): 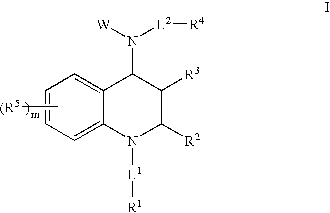
or a pharmaceutically acceptable salt or prodrug thereof, wherein
W is selected from the group consisting of aryl, heteroaryl, (C₁-C₈)alkyl and cyclo(C₃-C₈)alkyl;
L¹ is selected from the group consisting of C(O), SO₂ and (C₁-C₄)alkylene;
L² is selected from the group consisting of a single bond, C(O) and SO₂;
R¹ is selected from the group consisting of (C₁-C₈)alkyl, aryl, aryl(C₁-C₄)alkyl, aryl(C₁-C₄)alkoxy, aryl(C₁-C₄)alkenyl and heteroaryl;
R² and R³ are independently hydrogen or (C₁-C₈)alkyl;
R⁴ is selected from the group consisting of (C₁-C₈)alkyl, aryl(C₁-C₄)alkyl, cyclo(C₃-C₈)alkyl(C₁-C₄)alkyl, hydroxy(C₁-C₄)alkyl, (C₁-C₄)alkoxy(C₁-C₄)alkyl, amino(C₁-C₄)alkyl, (C₁-C₄)alkylamino(C₁-C₄)alkyl, di(C₁-C₄)alkylamino(C₁-C₄)alkyl, carboxy(C₁-C₄)alkyl, (C₁-C₄)alkoxycarbonyl(C₁-C₄)alkyl, carbamoyl(C₁-C₄)alkyl and carboxy(C₂-C₄)alkenyl;
each R⁵ is independently selected from the group consisting of halogen, (C₁-C₈)alkyl, (C₁-C₄)alkoxy, thio(C₁-C₄)alkoxy, amino, (C₁-C₄)alkylamino, di(C₁-C₄)alkylamino, halo(C₁-C₄)alkyl, halo(C₁-C₄)alkoxy, cyano, nitro, —CO₂R′, —CONR′R″, —C(O)R′, —OC(O)R′, —OC(O)NR′R″, —NR″C(O)R′, —NR″CO₂R′, —N(R′)C(O)NR″R′″, —NR′C(NH₂)═NR″, —S(O)R′, —SO₂R′, —SO₂NR′R″, —N₃ and —CH(Ph)₂;
optionally, two adjacent R⁵ groups may be combined to form a 5-, 6-, 7- or 8membered fused ring containing the carbon atoms to which they are attached and 0, 1 or 2 additional heteroatoms selected from N, O and S;
R′, R″ and R′″ are independently selected from the group consisting of hydrogen, (C₁-C₈)alkyl, aryl, aryl(C₁-C₄)alkyl and heteroaryl;
optionally, when R′ and R″ or R″ and R′″ are attached to the same nitrogen atom, R′ and R″ or R″ and R′″ may be combined to form a 5-, 6-, 7- or 8-membered ring containing the nitrogen atom to which they are attached and 0, 1 or 2 additional heteroatoms selected from N, O and S; and
the subscript m is 0, 1, 2, 3 or 4.

31. (Canceled)

32. The method of claim 30, wherein said compound modulates CRTH2.

33. The method of claim 32, wherein said compound is a CRTH2 antagonist.
